SEQUENCE LISTING (1 sequence)

SEQ ID NO: 1 - DNA, 30 bp, synthetic construct
  source 1..30: mol_type = other DNA; note = Description of Artificial Sequence: Synthetic oligonucleotide
  modified_base 1: mod_base = OTHER; note = thymidine phosphorothioate
  modified_base 2: mod_base = OTHER; note = cytidine phosphorothioate
  modified_base 3: mod_base = OTHER; note = guanosine phosphorothioate
  modified_base 4: mod_base = OTHER; note = adenosine phosphorothioate
  modified_base 5: mod_base = OTHER; note = adenosine phosphorothioate
  modified_base 6: mod_base = OTHER; note = cytidine phosphorothioate
  modified_base 7: mod_base = OTHER; note = guanosine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = cytidine phosphorothioate
  modified_base 11: mod_base = OTHER; note = guanosine phosphorothioate
  modified_base 12: mod_base = OTHER; note = adenosine phosphorothioate
  modified_base 13: mod_base = OTHER; note = adenosine phosphorothioate
  modified_base 14: mod_base = OTHER; note = cytidine phosphorothioate
  modified_base 15: mod_base = OTHER; note = guanosine phosphorothioate
  modified_base 16: mod_base = OTHER; note = thymidine phosphorothioate
  modified_base 17: mod_base = OTHER; note = thymidine phosphorothioate
  modified_base 18: mod_base = OTHER; note = cytidine phosphorothioate
  modified_base 19: mod_base = OTHER; note = guanosine phosphorothioate
  modified_base 20: mod_base = OTHER; note = adenosine phosphorothioate
  modified_base 21: mod_base = OTHER; note = adenosine phosphorothioate
  modified_base 22: mod_base = OTHER; note = cytidine phosphorothioate
  modified_base 23: mod_base = OTHER; note = guanosine phosphorothioate
  modified_base 24: mod_base = OTHER; note = thymidine phosphorothioate
  modified_base 25: mod_base = OTHER; note = thymidine phosphorothioate
  modified_base 26: mod_base = OTHER; note = cytidine phosphorothioate
  modified_base 27: mod_base = OTHER; note = guanosine phosphorothioate
  modified_base 28: mod_base = OTHER; note = adenosine phosphorothioate
  modified_base 29: mod_base = OTHER; note = adenosine phosphorothioate
tcgaacgttcgaacgttcgaacgttcgaat

(19) United States
(12) Patent Application Publication
(10) Pub. No.: US 2025/0101438 A1
(43) Pub. Date: Mar. 27, 2025

(54) METHODS OF TREATING IMMUNE DYSFUNCTION IN LIVER CANCER WITH TOLL-LIKE RECEPTOR AGONISTS

(71) Applicant: TriSalus Life Sciences, Inc., Westminster, CO (US)

(72) Inventors: Steven C. Katz, East Greenwich, RI (US); Bryan F. Cox, Muscatine, IA (US); Chandra C. Ghosh, Sharon, MA (US); Prajna Guha, Sharon, MA (US); Jason LaPorte, West Warwick, RI (US)

(73) Assignee: TriSalus Life Sciences, Inc., Westminster, CO (US)

(21) Appl. No.: 18/730,982

(22) PCT Filed: Jan. 20, 2023
(86) PCT No.: PCT/US2023/061035

§ 371 Date: Jul. 22, 2024

Related U.S. Application Data
(60) Provisional application No. 63/302,004, filed on Jan. 21, 2022.
(60) Provisional application No. 63/423,005, filed on Nov. 6, 2022.
(60) Provisional application No. 63/426,757, filed on Nov. 20, 2022.

(51) Int. Cl. C12N 15/117; A61K 31/7125; A61K 39/00; A61K 39/395; A61P 35/04

(52) U.S. Cl. CPC C12N 15/117; A61K 31/7125; A61K 39/39558; A61P 35/04; A61K 2039/54; A61K 2039/545; A61K 2039/55561; C12N 2310/17; C12N 2310/315

(57) ABSTRACT

Methods for treating liver immune dysfunction in a subject are provided. The liver immune dysfunction may be induced by at least one tumor resulting from metastasis in the liver or at least one primary liver cancer. An exemplary method comprises administering to the subject a toll-like receptor 9 (TLR9) agonist, for example, a TLR 9 agonist having the structure: 5′-TCG AAC GTT CGA ACG TTC GAA CGT TCG AAT-3. The TLR9 agonist is administered to the liver, for example, the TLR9 agonist may be administered to the liver using a locoregional therapy through the vasculature.

### PRIORITY CLAIM

This application claims priority to U.S. Provisional Application Ser. No. 63/302,004 entitled “Cancer Therapy Using Toll-Like Receptor Agonists” filed on Jan. 21, 2022, to U.S. Provisional Application Ser. No. 63/423,005 entitled “Cancer Therapy Using Toll-Like Receptor Agonists” filed on Nov. 6, 2022, and to U.S. Provisional Application Ser. No. 63/426,757 entitled “Cancer Therapy-Using Toll-Like Receptor Agonists” filed on Nov. 20, 2022, the entire contents of all of the applications identified above are hereby incorporated by reference herein.

### SEQUENCE LISTING

The present application contains a Sequence Listing which has been submitted electronically in XML format and is hereby incorporated by reference in its entirety. Said XML copy, created on Jan. 20, 2023, is named A372-509_WO_SL.xml and is 18,185 bytes in size.

### FIELD OF THE INVENTION

The present disclosure relates generally to methods of treating cancer, in particular, liver cancer, and methods of delivering toll-like receptor (TLR) agonists to solid tumors in the liver using a locoregional therapy through the vasculature.

### BACKGROUND OF THE INVENTION

Cancer is a devastating disease that involves the unchecked growth of cells, which may result in the growth of solid tumors in a variety of organs such as the skin, liver, and pancreas. Tumors may first present in any number of organs or may be the result of metastases or spread from other locations.

The liver is a unique organ which is intrinsically immunosuppressive and drives the programming and expansion of suppressive cells such as myeloid derived suppressor cells (MDSCs). In this regard, MDSCs expand in response to malignancy. MDSCs also drive expansion of other suppressor cell types such as T regulatory cells (Tregs), tumor-associated macrophages (TAMs), and cancer-associated fibroblasts (CAFs). MDSCs may downregulate immune cells and interfere with the effectiveness of immunotherapeutics. Further, high MDSC levels generally predict poor outcomes in cancer patients.

Specific delivery challenges and immunosuppressive pathways may limit immunotherapy success in the liver. Therefore, there remains a need for a safe and effective therapy for the treatment of liver immune dysfunction.

### SUMMARY OF THE INVENTION

The present application relates to methods of treating liver immune dysfunction using a therapeutically effective amount of a toll-like receptor (TLR) agonist. For example, the present application provides a method of treating liver immune dysfunction comprising administering to a subject in need thereof a therapeutically effective amount of a toll-like receptor 9 agonist having the structure: 5′-TCG AAC GTT CGA ACG TTC GAA CGT TCG AAT-3′ (SEQ ID NO: 1). The liver immune dysfunction is induced by at least one tumor resulting from metastasis in the liver or at least one primary liver cancer.

In one aspect, the present application relates to a method of treating liver immune dysfunction comprising administering a TLR agonist through a device by hepatic arterial infusion (HAI). According to another example, the treatment of liver immune dysfunction comprises administering a TLR agonist through a device by portal vein infusion (PVI). For example, the TLR9 agonist may be administered through a catheter device. The catheter device comprise a one-way valve that responds dynamically to local pressure and/or flow changes. In another example, the TLR9 agonist is administered through the catheter device via pressure-enabled drug delivery (PEDD).

In some examples, the liver immune dysfunction can be induced and/or a result of a solid tumor in the liver, such as a tumor that is the metastases of uveal melanoma, or a result of at least one primary liver cancer, such as hepatocellular carcinoma or intrahepatic cholangiocarcinoma.

In some examples, the TLR agonist is administered through PEDD, which includes the administration of a therapeutic through a device, such as a catheter device. In some examples, the catheter device comprises a one-way valve that responds dynamically to local pressure changes. In some examples, the catheter device generates, causes, and/or contributes to a net increase in fluid pressure within the vessel and/or target tissue or tumor. In some examples, the catheter device generates, causes, and/or contributes to a net decrease in fluid pressure within the vessel and/or target tissue or tumor. In some examples, the catheter device generates, causes, and/or contributes to first a decrease, then an increase in fluid pressure within the vessel and/or target tissue or tumor.

In some examples, the TLR agonist is administered through a pressure-enabled device, such as one that modulates vascular pressure.

In some examples, the amount of TLR agonist administered is in the range of about 0.01-20 mg, or is selected from the group consisting of 2 mg, 4 mg, or 8 mg.

In some examples, the TLR agonist is administered for a period of time of about 10-200 minutes. In another embodiment, the TLR agonist is administered for a period of time of about 10-60 minutes. In another embodiment, the TLR agonist is administered for a period of time of about 25 minutes.

In some examples, the administration the TLR9 agonist results in changes in gene expression within the metastasis in the liver. For example, the changes in the gene expression include activation of the immune cells in normal liver tissue and migration of the activated immune cells into the at least one tumor. In another example, the changes in the gene expression include at least one of: increased TLR signaling; increased leukocytes; increased exhausted CD8 T cells; induction of Th1 programming; reduction of Th2 programming; increased T cell receptor and T cell co-stimulatory signaling; increased IL9, IL15, CCL7; B cell activation; induction of mast cells; induction of NK cells; induction of IFNγ; increased interferon signaling; increased chemokine signaling; increased IL6 in the liver without an increase in the blood; decreased M2 macrophages; increased M1 macrophages; decreased in MDSC; decreased angiogenesis and VEGF; and decreased fatty acid oxidation.

In some examples, the TLR agonist is administered in combination with one or more checkpoint inhibitors. In some examples, the checkpoint inhibitors are administered systemically, either concurrently, before, or after the administration of the TLR agonist. In some examples, the checkpoint inhibitors include at least one of nivolumab, pembrolizumab, and ipilimumab. In some examples, the administration of the TLR9 agonist in combination with the one or more checkpoint inhibitors may result in a decrease of one of circulating tumor cell levels and circulating tumor DNA levels. In certain aspects, the one or more checkpoint inhibitors is administered intraperitoneally or subcutaneously.

In some examples, administration of the TLR agonist comprises a dosing regimen comprising cycles. In some examples, one or more of the cycles comprise the administration of the TLR agonist via a catheter device by HAI followed by the systemic administration of one or more checkpoint inhibitors. Specifically, the checkpoint inhibitors include at least one of nivolumab, pembrolizumab, and ipilimumab.

In some examples, the administration of a TLR agonist through an intravascular device to the liver results in a reduction of myeloid-derived suppressor cells (MDSC) or the functional alteration of MDSCs to limit immunosuppression. In some examples, the administration of a TLR agonist through an intravascular device to the liver results in antitumor effects.

In some examples, the TLR agonist is a TLR9 agonist, more particularly, a class C TLR9 agonist. In some examples, the class C TLR9 agonist is SD-101.

In another example, the methods of the present application include a method for treating liver immune dysfunction and tumor-induced liver immune dysfunction, wherein the administration of the TLR9 agonist, more specifically, the class C TLR9 agonist, results in at least one of the following changes in gene expression within liver metastases (LM): activation of immune cells in the normal liver (non-tumor liver in the same patient), with migration into liver metastases; activation and mobilization of peripheral blood immune cells (T, NK, B, cytotoxic T, CD4 Th1 T, exhausted T, macrophages); increased TLR signaling; increased leukocytes (CD45+); increased exhausted CD8 T cells (very important for checkpoint responsiveness); induction of Th1 programming; reduction of Th2 programming; increased T cell receptor and T cell co-stimulatory signaling; increased IL9, IL15, CCL7; B cell activation; induction of mast cells; induction of NK cells; induction of IFNg and related genes; increased interferon signaling; increased chemokine signaling; increased IL6 in the liver without an increase in the blood; decreased M2 macrophages; increased M1/activated macrophages and associated genes: CD68, CD86 dendritic cell activation and migration to draining lymph nodes; decreases in MDSC and associated genes: IDO1, TOD2, ARG-1, NOS2, TIMN/D4; decreased angiogenesis and VEGF; decreased fatty acid oxidation (a preferred MDSC program).

In another example, the methods of the present application include a method for administering a TLR9 agonist, in particular, a class C TLR9 agonist, and more particularly, in combination with a checkpoint inhibitor to a patient, wherein the administration of the TLR9 agonist in combination with the checkpoint inhibitor results in a decrease in circulating tumor cell levels and/or circulating tumor DNA levels. The patient may have liver immune dysfunction and/or tumor-induced liver immune dysfunction. For example, the patient may have metastatic uveal melanoma in the liver, hepatocellular carcinoma, or intrahepatic cholangiocarcinoma.

These and other objects, features, and advantages of the exemplary embodiments of the present disclosure will become apparent upon reading the following detailed description of the exemplary embodiments of the present disclosure, when taken in conjunction with the entire specification.

### BRIEF DESCRIPTION OF THE DRAWINGS

Further objects, features and advantages of the present disclosure will become apparent from the following detailed description taken in conjunction with the accompanying Figures showing illustrative examples of the present disclosure.

FIG. 1 illustrates the structure of SD-101.

FIG. 2A provides in vitro experimental data demonstrating the effect of a TLR4 agonist, a TLR7 agonist, a TLR9A agonist, a TLR9B agonist and SD-101 as compared to non-treated control on amount of total MDSCs induced by treatment of human PBMCs harvested from healthy donors and treated with IL6 and GMCSF, according to Example 1.

FIG. 2B provides in vitro experimental data demonstrating the effect of a TLR4 agonist, a TLR7 agonist, a TLR9A agonist, a TLR9B agonist and SD-101 as compared to non-treated control on amount of M-MDSC and G-MDSC induced by treatment of human PBMCs harvested from healthy donors and treated with IL6 and GMCSF, according to Example 1.

FIG. 2C provides in vitro experimental data demonstrating the effect of a TLR4 agonist, a TLR7 agonist, a TLR9A agonist, a TLR9B agonist and SD-101 as compared to non-treated control on amount of M1 macrophages induced by treatment of human PBMCs harvested from healthy donors and treated with IL6 and GMCSF, according to Example 1.

FIG. 2D provides in vitro experimental data demonstrating the effect of a TLR4 agonist, a TLR7 agonist, a TLR9A agonist, a TLR9B agonist and SD-101 as compared to non-treated control on amount of M2 macrophages induced by treatment of human PBMCs harvested from healthy donors and treated with IL6 and GMCSF, according to Example 1.

FIG. 2E provides an image of in vitro experiment demonstrating the effect of a TLR9 agonist as compared to non-treated control on human MDSC induced by treatment of human PBMCs harvested from healthy donors and treated with IL6 and GMCSF, according to Example 1.

FIG. 3A provides in vitro experimental data demonstrating the effect of a TLR4 agonist, a TLR7 agonist, a TLR9A agonist, a TLR9B agonist and SD-101 as compared to non-treated control on amount of MDSC (CD11b+Gr-1+) induced by treatment of murine bone marrow cells treated with GMCSF, according to Example 2.

FIG. 3B provides in vitro experimental data demonstrating the effect of a TLR4 agonist, a TLR7 agonist, a TLR9A agonist, a TLR9B agonist and SD-101 as compared to non-treated control on amount of macrophages (CD11b+F4/CD80+) induced by treatment of murine bone marrow cells treated with GMCSF, according to Example 2.

FIG. 3C provides in vitro experimental data demonstrating the effect of a TLR4 agonist, a TLR7 agonist, a TLR9A agonist, a TLR9B agonist and SD-101 as compared to non-treated control on amount of M1 macrophages (CD11b+F4/CD80+CD38+) induced by treatment of murine bone marrow cells treated with GMCSF, according to Example 2.

FIG. 4 illustrates changes in liver metastasis gene expression four weeks following a first cycle of three weekly SD-101 infusions via HAI and PEDD at 2 mg dosing, as measured by a NanoString® immune cell panel, according to Example 3.

FIG. 5 illustrates changes in liver metastasis gene expression four weeks following a first cycle of three weekly SD-101 infusions via HAI and PEDD at 2 mg dosing, as measured by a NanoString® myeloid panel, according to Example 3.

FIG. 6 illustrates changes in liver metastasis gene expression four weeks following a first cycle of three weekly SD-101 infusions via HAI and PEDD at 2 mg dosing, as measured by a NanoString® myeloid cell panel, according to Example 3.

FIG. 7 illustrates changes in liver metastasis gene expression four weeks following a first cycle of three weekly SD-101 infusions via HAI and PEDD at 2 mg dosing, as measured by a NanoString® metabolic panel, according to Example 3.

FIG. 8 illustrates changes in liver metastasis gene expression four weeks following a first cycle of three weekly SD-101 infusions via HAI and PEDD at 2 mg dosing, as measured by a NanoString® myeloid panel, according to Example 3.

FIG. 9 illustrates changes in normal adjacent liver tissue gene expression four weeks following a first cycle of three weekly SD-101 infusions via HAI and PEDD at 2 mg dosing, as measured by a NanoString® myeloid panel, according to Example 3.

FIG. 10 illustrates changes in PBMC gene expression over two cycles of three weekly SD-101 infusions each via HAI and PEDD at 2 mg dosing, as measured by NanoString® myeloid panel, according to Example 3.

FIG. 11A provides experimental data demonstrating that TLR9 stimulation with SD-101 inhibits MDSC generation from human PBMCs treated with IL6 and GMCSF for 7 days, according to Example 4.

FIG. 11B provides experimental data demonstrating that TLR9 stimulation with SD-101 inhibits MDSC generation from human PBMCs treated with IL6 and GMCSF for 2 days, according to Example 4.

FIG. 11C provides experimental data demonstrating that TLR9 stimulation with SD-101 enhances M1 macrophage production from human PBMCs treated with IL6 and GMCSF for 7 days, according to Example 4.

FIG. 11D provides experimental data demonstrating that TLR9 stimulation with SD-101 enhances M1 macrophage production from human PBMCs treated with IL6 and GMCSF for 2 days, according to Example 4.

FIG. 12A provides experimental data demonstrating the effect of a TLR4 agonist, a TLR7 agonist, a TLR9B agonist and SD-101 as compared to non-treated control on amount of MDSC (CD33⁺CD11b⁺HLADR⁻) induced by treatment of human PBMCs harvested from healthy donors and treated with IL6 and GMCSF, according to Example 5.

FIG. 12B provides experimental data demonstrating the effect of a TLR4 agonist, a TLR7 agonist, a TLR9B agonist and SD-101 as compared to non-treated control on amount of M1 macrophages (CD14⁺CD86⁺) induced by treatment of human PBMCs harvested from healthy donors and treated with IL6 and GMCSF, according to Example 5.

FIG. 12C provides experimental data demonstrating the effect of a TLR4 agonist, a TLR7 agonist, a TLR9B agonist and SD-101 as compared to non-treated control on amount of M/G-MDSCs (M/G CD14⁺/CD15⁺MDSCs) induced by treatment of human PBMCs harvested from healthy donors and treated with IL6 and GMCSF, according to Example 5.

FIG. 12D provides experimental data demonstrating the effect of a TLR4 agonist, a TLR7 agonist, a TLR9B agonist and SD-101 as compared to non-treated control on amount of monocytic dendritic cells (CD14⁺CD11c⁺CD123⁻) induced by treatment of human PBMCs harvested from healthy donors and treated with IL6 and GMCSF, according to Example 5.

FIG. 12E provides experimental data demonstrating the effect of a TLR4 agonist, a TLR7 agonist, a TLR9B agonist and SD-101 as compared to non-treated control on amount of murine bone marrow MDSCs from mouse bone marrow (BM) treated in the absence of murine GMCSF, according to Example 5.

FIG. 12F provides experimental data demonstrating the effect of a TLR4 agonist, a TLR7 agonist, a TLR9B agonist and SD-101 as compared to non-treated control on amount of murine bone marrow MDSCs from mouse bone marrow (BM) treated in the presences of murine GMCSF, according to Example 5.

FIG. 13A shows a volcano plot illustrating gene expression of human PBMCs after treatment with TLR9B agonist vs. SD-101 (TLR9C agonist) for 2 days, as determined using NanoString® mRNA analysis, according to Example 6.

FIG. 13B provides experimental data demonstrating the effect of a TLR9B agonist and SD-101 as compared to non-treated control on amount of MDSC (CD33⁺CD11b⁺HLADR⁻) induced by treatment of human PBMCs harvested from healthy donors and treated with IL6 and GMCSF, according to Example 6.

FIG. 13C provides experimental data demonstrating the effect of a TLR9B agonist and SD-101 as compared to non-treated control on amount of M1 macrophages (CD14⁺CD86⁺) induced by treatment of human PBMCs harvested from healthy donors and treated with IL6 and GMCSF, according to Example 6.

FIG. 13C provides experimental data demonstrating the effect of a TLR9B agonist and SD-101 as compared to non-treated control on amount of M1 macrophages (CD14⁺CD86⁺) induced by treatment of human PBMCs harvested from healthy donors and treated with IL6 and GMCSF, according to Example 6.

FIG. 13D shows pathway scores for immunoregulatory pathways affected by TLR9B vs. SD-101 as determined by nSolver.4 software using data generated from the NanoString® analysis of FIG. 13A.

FIG. 13E shows qRT-PCR data for PD-L1 expression of the same samples analyzed in FIG. 13A.

FIG. 13F shows qRT-PCR data for IFNγ expression of the same samples analyzed in FIG. 13A.

FIG. 13G shows qRT-PCR data for IP-10 expression of the same samples analyzed in FIG. 13A.

FIG. 14 illustrates changes in SD-101 levels in plasma following 4 mg dosing of single agent SD-101 to human patients, after each of six doses administered over two cycles, according to Example 7.

FIG. 15 illustrates changes in serum IFNγ following increasing dosing levels of single agent SD-101, and SD-101 in combination with a checkpoint inhibitor, to human patients after each of three doses administered over a first cycle, according to Example 7.

FIG. 16 illustrates changes in monocytic MDSC levels within tumors following varying dosing levels of single agent SD-101 to human patients, according to Example 7.

FIG. 17 illustrates changes in circulating tumor cells following 2 mg dosing of SD-101 with a checkpoint inhibitor to human patients, according to Example 7.

FIG. 18 illustrates changes in circulating tumor DNA following 2 mg dosing of SD-101 with a checkpoint inhibitor to 5 of the human patients of FIG. 17, according to Example 7.

FIG. 19A illustrates a change in the tumor progression of the mouse liver metastasis in response to PEDD administration of SD-101 in combination with anti-PD-1 antibody delivered either IP or SQ as compared to Vehicle, according to Example 8.

FIG. 19B illustrates changes in the tumor burden of the mouse liver metastasis in response to PEDD administration of SD-101 in combination with anti-PD-1 antibody delivered either IP or SQ as compared to Vehicle at 10 days after treatment, according to Example 8.

FIG. 19C provides images captured by the IVIS over different time points after initiating treatment by PEDD administration of SD-101 in combination with anti-PD-1 antibody delivered either IP or SQ as compared to Vehicle, according to Example 8.

FIG. 19D provides images captured of harvested liver at after 10 days of treatment with PEDD administration of SD-101 in combination with anti-PD-1 antibody delivered either IP or SQ as compared to Vehicle, according to Example 8.

FIG. 20A illustrates changes in MDSCs present in the LM in response to PEDD administration of SD-101 in combination with anti-PD-1 antibody delivered either IP or SQ as compared to Vehicle at 10 days after treatment, according to Example 8.

FIG. 20B illustrates changes in the B (B220⁺) cells of the isolated CD45⁺ cells from liver metastasis in response to PEDD administration of SD-101 in combination with anti-PD-1 antibody delivered either IP or SQ as compared to Vehicle at 10 days after treatment, according to Example 8.

FIG. 20C illustrates changes in the T (CD3⁺) cells of the isolated CD45⁺ cells from liver metastasis in response to PEDD administration of SD-101 in combination with anti-PD-1 antibody delivered either IP or SQ as compared to Vehicle at 10 days after treatment, according to Example 8.

FIG. 20D illustrates changes in the M1 and M2 macrophages of the isolated CD45⁺ cells from liver metastasis. M1(F4/80⁺CD38⁺Egr2⁻), and M2 (F4/80OCD38⁻Egr2⁺) macrophages in the tumor microenvironment, as quantified by FC, in response to PEDD administration of SD-101 in combination with anti-PD-1 antibody delivered either IP or SQ as compared to Vehicle at 10 days after treatment, according to Example 8.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended paragraphs.

### DETAILED DESCRIPTION

The following description of embodiments provides non-limiting representative examples referencing numerals to particularly describe features and teachings of different aspects of the invention. The embodiments described should be recognized as capable of implementation separately, or in combination, with other embodiments from the description of the embodiments. A person of ordinary skill in the art reviewing the description of embodiments should be able to understand the different described aspects of the invention. The description of embodiments should facilitate understanding of the invention to such an extent that other implementations, not specifically covered but within the knowledge of a person of skill in the art having read the description of embodiments, would be understood to be consistent with application of the invention.

Unless defined otherwise, all technical and scientific terms used herein have the same meaning commonly understood to one of ordinary skill in the art to which this application pertains. Otherwise, certain terms used herein have the meanings as set in the specification. All patents, published patent applications and publications cited herein are incorporated by reference as if set forth fully herein.

The singular forms “a,” “an,” and, “the” include plural references unless the context clearly dictates otherwise.

Unless otherwise stated, any numerical value, such as a concentration or a concentration range described herein, are to be understood as being modified in all instances by the term “about.” The numerical value may include ±1%, ±2%, ±3%, ±4%, or ±5% of the recited value. As used herein, the use of a numerical range expressly includes all possible subranges, all individual numerical values within that range, including integers within such ranges and fractions of the values unless the context clearly indicates otherwise.

The term “subject” or “patient” as used herein refers to an animal, and preferably a mammal, and more preferably, a human. Examples of subjects include humans, and may also include other animals such as rats, mice and pigs. In one specific aspect, the subject is a human.

As used herein, the terms “treat,” “treating,” and “treatment” are all intended to refer to an amelioration or reversal of at least one measurable physical parameter related to a disease, disorder, or condition. The terms “treat,” “treating,” and “treatment,” can also refer to causing regression, preventing the progression, or at least slowing down the progression of the disease, disorder, or condition. In a particular example, “treat,” “treating,” and “treatment” refer to prevention of the recurrence of the disease, disorder, or condition. In another example, “treat,” “treating,” and “treatment” refer to an increase in the survival of a subject having the disease, disorder, or condition. In a particular example, “treat,” “treating,” and “treatment” refer to elimination of the disease, disorder, or condition in the subject.

The term “therapeutically effective amount” as used herein refers to that amount of active compound or pharmaceutical agent that elicits a desired biological or medicinal response in a tissue system, animal or human, which includes improvement of one or more biomarkers for the disease, disorder or condition being treated or reduction of the severity of one or more of the symptoms of the disease, disorder or condition being treated, for example, slowing of tumor development and metastasis in a patient.

The term “agonist” as used herein refers to a compound that binds to a receptor that then increases, facilitates, sensitizes, or up-regulates the receptor.

The term “antagonist” as used herein refers to a compound that binds to a receptor that blocks or attenuates the receptor's response to an agonist.

The term “pharmaceutically acceptable salt” refers to a salt of a compound which are known to be non-toxic and are commonly used in the pharmaceutical arts. In some examples, the pharmaceutically acceptable salt of a compound retains its biological effectiveness and is not biologically or otherwise undesirable.

As used herein, a “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an active compound or pharmaceutical agent and that is compatible therewith.

### Toll-Like Receptor Agonists

Toll-like receptors are pattern recognition receptors that can detect microbial pathogen-associated molecular patterns (PAMPs). TLR stimulation, such as TLR9 stimulation, can not only provide broad innate immune stimulation, but can also specifically address the dominant drivers of immunosuppression in the liver. TLR1-10 are expressed in humans and recognize a diverse variety of microbial PAMPs. In this regard, TLR9 can respond to unmethylated CpG-DNA, including microbial DNA. CpG refers to the motif of a cytosine and guanine dinucleotide. TLR9 is constitutively expressed in B cells, plasmacytoid dendritic cells (pDCs), activated neutrophils, monocytes/macrophages, T cells, and MDSCs. Further, human MDSCs express TLR9 on their surface. Further, TLR9 and related endosomal protein TLR7 are expressed in human liver metastases tissue. TLR9 is also expressed in non-immune cells, including keratinocytes and gut, cervical, and respiratory epithelial cells. TLR9 can bind to its agonists within endosomes. Signaling may be carried out through MYD88/IkB/NfκB to induce pro-inflammatory cytokine gene expression. A parallel signaling pathway through IRF7 induces type 1 and 2 interferons (e.g., IFN-α, IFN-γ, etc.) which stimulate adaptive immune responses. Further, TLR9 agonists can induce cytokine and IFN production and functional maturation of antigen presenting dendritic cells.

According to an example, a TLR9 agonist can reduce and reprogram MDSCs. In this regard, reducing, altering, or eliminating MDSCs is thought to improve the ability of the host's immune system to attack the cancer as well as the ability of the immunotherapy to induce more beneficial therapeutic responses. In an example, TLR9 agonists may convert MDSCs into immunostimulatory M1 macrophages, convert immature dendritic cells to mature dendritic cells, and expand effector T cells to create a responsive tumor microenvironment to promote anti-tumor activity.

According to an example, synthetic CpG-oligonucleotides (CPG-ONs) mimicking the immunostimulatory nature of microbial CpG-DNA can be developed for therapeutic use. According to an example, the oligonucleotide is an oligodeoxynucleotide (ODN). There are a number of different CpG-ODN class types, e.g. Class A, Class B, Class C, Class P, and Class S, which share certain structural and functional features. In this regard, Class A type CPG-ODNs (or CPG-A ODNs) are associated with pDC maturation with little effect on B cells as well as the highest degree of IFNα induction; Class B type CPG-ODNs (or CPG-B ODNs) strongly induce B-cell proliferation, activate pDC and monocyte maturation, NK cell activation, and inflammatory cytokine production; and Class C type CPG-ODNs (or CPG-C ODNs) can induce B-cell proliferation and IFN-α production.

Further, according to an example, CPG-C ODNs can be associated with the following attributes: (i) unmethylated dinucleotide CpG motifs, (ii) juxtaposed CpG motifs with flanking nucleotides (e.g., AACGTTCGAA), (iii) a complete phosphorothioate (PS) backbone that links the nucleotides (as opposed to the natural phosphodiester (PO) backbones found in bacterial DNA), and (iv) a self-complimentary, palindromic sequence (e.g. AACGTT). In this regard, CPG-C ODNs may bind themselves due to their palindromic nature, thereby producing double-stranded duplex (e.g., dimer) or hairpin structures.

Further, according to an example, the CPG-C ODNs can include one or more 5′-TCG trinucleotides wherein the 5′-T is positioned 0, 1, 2, or 3 bases from the 5′-end of the oligonucleotide, and at least one palindromic sequence of at least 8 bases in length comprising one or more unmethylated CG dinucleotides. The one or more 5′-TCG trinucleotide sequence may be separated from the 5′-end of the palindromic sequence by 0, 1, or 2 bases or the palindromic sequence may contain all or part of the one or more 5′-TCG trinucleotide sequence. In an example, the CpG-C ODNs are 12 to 100 bases in length, preferably 12 to 50 bases in length, preferably 12 to 40 bases in length, or preferably 12-30 bases in length. In an example, the CpG-C ODN is 30 bases in length. In an example, the ODN is at least (lower limit) 12, 13, 14, 15, 16, 17, 18, 19, 20, 21, 22, 23, 24, 25, 26, 27, 28, 29, 30, 32, 34, 36, 38, 40, 50, 60, 70, 80, or 90 bases in length. In an example, the ODN is at most (upper limit) 100, 90, 80, 70, 60, 50, 49, 48, 47, 46, 45, 44, 43, 42, 41, 40, 39, 38, 37, 36, 35, 34, 33, 32, 31, or 30 bases in length.

In an example, the at least one palindromic sequence is 8 to 97 bases in length, preferably 8 to 50 bases in length, or preferably 8 to 32 bases in length. In a specific example, the at least one palindromic sequence is 8 bases in length. In an example, the at least one palindromic sequence is at least (lower limit) 8, 10, 12, 14, 16, 18, 20, 22, 24, 26, 28, or 30 bases in length. In an example, the at least one palindromic sequence is at most (upper limit) 50, 48, 46, 44, 42, 40, 38, 36, 34, 32, 30, 28, 26, 24, 22, 20, 18, 16, 14, 12 or 10 bases in length.

In an example, the CpG-C ODN can comprise the sequence of SEQ ID NO: 1, as indicated below.

According to an example, the CpG-C ODN can comprise the SD-101 or a pharmaceutically acceptable salt thereof. SD-101 is a 30-mer phosphorothioate oligodeoxynucleotide, having the following sequence:

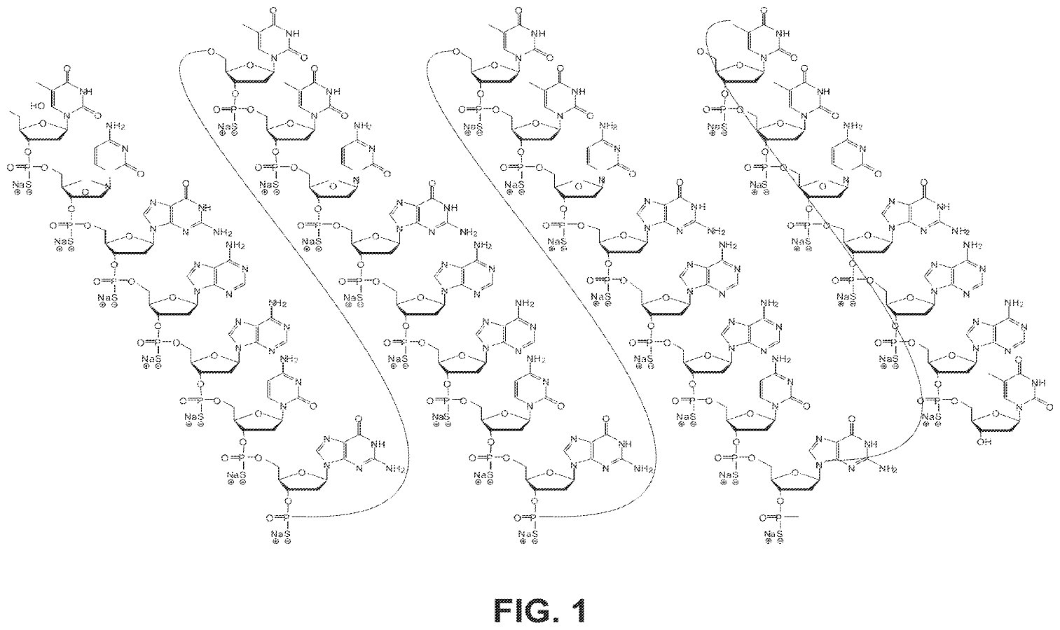

(SEQ ID NO: 1)
5′-TCG AAC GTT CGA ACG TTC GAA CGT TCG AAT-3′,

wherein the entire 30-mer sequence is linked by phosphorothioate bonds.SD-101 may be in the form of a free acid. In particular, the SD-101 drug substance is isolated as a sodium salt. The structure of the sodium salt of SD-101 is illustrated in FIG. 1.

The molecular formula of SD-101 in a free acid form is C₂₉₃ H₃₆₉ N₁₁₂ O₁₄₉ P₂₉ S₂₉ and the molecular mass of the SD-101 in the free acid form is 9672 Daltons. The molecular formula of the sodium salt of SD-101, as shown in FIG. 1, is C₂₉₃ H₃₄₀ N₁₁₂ O₁₄₉ P₂₉ S₂₉ Na₂₉ and the molecular mass of the sodium salt of SD-101 is 10,309 Daltons.

Further, according to an example, the CPG-C ODN sequence can correspond to SEQ ID NO: 172 as described in U.S. Pat. No. 9,422,564, which is incorporated by reference herein in its entirety.

In an example, the CpG-C ODN can comprise a sequence that has at least 75% homology to any of the foregoing, such as SEQ ID NO:1.

According to another example the CPG-C ODN sequence can correspond to any one of the other sequences described in U.S. Pat. No. 9,422,564. Further, the CPG-C ODN sequence can also correspond to any of the sequences described in U.S. Pat. No. 8,372,413, which is also incorporated by reference herein in its entirety.

According to an example, any of the CPG-C ODNs discussed herein may be present in their pharmaceutically acceptable salt forms. Suitable pharmaceutically acceptable salts of any of the CPG-C ODNs may include organic and inorganic bases. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In an example, the CpG-C ODNs are in the ammonium, sodium, lithium, or potassium salt forms. In one preferred example, the CpG-C ODNs are in the sodium salt form. The CpG-C ODN may be provided in a pharmaceutical solution comprising one or more pharmaceutically acceptable excipients. Alternatively, the CpG-C ODN may be provided as a lyophilized solid, which is subsequently reconstituted in sterile water, saline or a pharmaceutically acceptable buffer before administration.

Pharmaceutically acceptable excipients of the present disclosure include, for instance, solvents, bulking agents, buffering agents, tonicity adjusting agents, and preservatives. In an example, the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent). The pharmaceutical compositions of the present disclosure are suitable for parenteral and/or percutaneous administration.

In an example, the pharmaceutical compositions comprise an aqueous vehicle as a solvent. Suitable vehicles include for instance sterile water, saline solution, phosphate buffered saline, and Ringer's solution. In an example, the composition is isotonic.

The pharmaceutical compositions may comprise a bulking agent. Bulking agents are particularly useful when the pharmaceutical composition is to be lyophilized before administration. In an example, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some examples, the buffering agent maintains the pH of the composition within a range of 4 to 9. In an example, the pH is greater than (lower limit) 4, 5, 6, 7 or 8. In some examples, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin, and mannitol.

The pharmaceutical compositions may comprise a preservative. Suitable preservatives include for instance antioxidants and antimicrobial agents. However, in an example, the pharmaceutical composition is prepared under sterile conditions and is in a single use container, and thus does not necessitate inclusion of a preservative.

Table 1 describes an exemplary batch formula for SD-101 Drug Product—16 g/L:

TABLE 1

| | | Concen- | Amount | Exemplary Lot |
|---|---|---|---|---|
| Ingredient | Grade | tration | per batch | Batch Size 2.224 L |
| SD-101 Drug | GMP | 1.6% | 16.00 g | 35.584 g¹ |
| Substance* | | | | |
| Sodium | USP/NF, EP | 1.02% | 1.02 g | 2.268 g |
| phosphate, | | | | |
| dibasic anhydrous | | | | |
| Sodium | USP | 0.34% | 0.34 g | 0.747 g |
| phosphate, | | | | |
| monobasic | | | | |
| anhydrous | | | | |
| Sodium chloride | USP/NF, EP | 7.31% | 7.31 g | 16.257 g |
| Sterile Water for | USP/NF, EP | QS | QS | 2.224 L (kg) (QS) |
| Injection | | | | |

¹Quantity based upon measured content in solution (to exclude moisture present in lyophilized powder)
*SD-101 Drug Substance in Table 1 reflects the totality of all oligonucleotide content, including SD-101.

In some example, the unit dose strength may include from about 0.1 mg/mL to about 20 mg/mL. In one example, the unit dose strength of SD-101 is 13.4 mg/mL.

CpG-C ODNs may contain modifications. Suitable modifications can include but are not limited to, modifications of the 3′OH or 5′OH group, modifications of the nucleotide base, modifications of the sugar component, and modifications of the phosphate group. Modified bases may be included in the palindromic sequence as long as the modified base(s) maintains the same specificity for its natural complement through Watson-Crick base pairing (e.g., the palindromic portion of the CpG-C ODN remains self-complementary). Examples of modifications of the 5′OH group can include biotin, cyanine 5.5, the cyanine family of dyes, Alexa Fluor 660, the Alexa Fluor family of dyes, IRDye 700, IRDye 800, IRDye 800CW, and the IRDye family of dyes.

CpG-C ODNs may be linear, may be circular or include circular portions and/or a hairpin loop. CpG-C ODNs may be single stranded or double stranded. CpG-C ODNs may be DNA, RNA or a DNA/RNA hybrid.

CpG-C ODNs may contain naturally-occurring or modified, non-naturally occurring bases, and may contain modified sugar, phosphate, and/or termini. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. In an example, CpG-C ODNs have only phosphorothioate linkages, only phosphodiester linkages, or a combination of phosphodiester and phosphorothioate linkages.

Sugar modifications known in the field, such as 2′-alkoxy-RNA analogs, 2′-amino-RNA analogs, 2′-fluoro-DNA, and 2′-alkoxy- or amino-RNA/DNA chimeras and others described herein, may also be made and combined with any phosphate modification. Examples of base modifications include but are not limited to addition of an electron-withdrawing moiety to C-5 and/or C-6 of a cytosine of the CpG-C ODN (e.g., 5-bromocytosine, 5-chlorocytosine, 5-fluorocytosine, 5-iodocytosine) and C-5 and/or C-6 of a uracil of the CpG-C ODN (e.g., 5-bromouracil, 5-chlorouracil, 5-fluorouracil, 5-iodouracil). As noted above, use of a base modification in a palindromic sequence of a CpG-C ODN should not interfere with the self-complementarity of the bases involved for Watson-Crick base pairing. However, outside of a palindromic sequence, modified bases may be used without this restriction. For instance, 2′-O-methyl-uridine and 2′-O-methyl-cytidine may be used outside of the palindromic sequence, whereas, 5-bromo-2′-deoxycytidine may be used both inside and outside the palindromic sequence. Other modified nucleotides, which may be employed both inside and outside of the palindromic sequence include 7-deaza-8-aza-dG, 2-amino-dA, and 2-thio-dT.

Duplex (i.e., double stranded) and hairpin forms of most ODNs are often in dynamic equilibrium, with the hairpin form generally favored at low oligonucleotide concentration and higher temperatures. Covalent interstrand or intrastrand cross-links increase duplex or hairpin stability, respectively, towards thermal-, ionic-, pH-, and concentration-induced conformational changes. Chemical cross-links can be used to lock the polynucleotide into either the duplex or the hairpin form for physicochemical and biological characterization. Cross-linked ODNs that are conformationally homogeneous and are “locked” in their most active form (either duplex or hairpin form) could potentially be more active than their uncross-linked counterparts. Accordingly, some CpG-C ODNs of the present disclosure can contain covalent interstrand and/or intrastrand cross-links.

The techniques for making polynucleotides and modified polynucleotides are known in the art. Naturally occurring DNA or RNA, containing phosphodiester linkages, may be generally synthesized by sequentially coupling the appropriate nucleoside phosphoramidite to the 5′-hydroxy group of the growing ODN attached to a solid support at the 3′-end, followed by oxidation of the intermediate phosphite triester to a phosphate triester. Using this method, once the desired polynucleotide sequence has been synthesized, the polynucleotide is removed from the support, the phosphate triester groups are deprotected to phosphate diesters and the nucleoside bases are deprotected using aqueous ammonia or other bases.

The CpG-C ODN may contain phosphate-modified oligonucleotides, some of which are known to stabilize the ODN. Accordingly, some examples include stabilized CpG-C ODNs. The phosphorous derivative (or modified phosphate group), which can be attached to the sugar or sugar analog moiety in the ODN, can be a monophosphate, diphosphate, triphosphate, alkylphosphonate, phosphorothioate, phosphorodithioate, phosphoramidate or the like.

CpG-C ODNs can comprise one or more ribonucleotides (containing ribose as the only or principal sugar component), deoxyribonucleotides (containing deoxyribose as the principal sugar component), modified sugars or sugar analogs. Thus, in addition to ribose and deoxyribose, the sugar moiety can be pentose, deoxypentose, hexose, deoxyhexose, glucose, arabinose, xylose, lyxose, and a sugar analog cyclopentyl group. The sugar can be in pyranosyl or in a furanosyl form. In the CpG-C oligonucleotide, the sugar moiety is preferably the furanoside of ribose, deoxyribose, arabinose or 2′-0-alkylribose, and the sugar can be attached to the respective heterocyclic bases in either anomeric configuration. The preparation of these sugars or sugar analogs and the respective nucleosides wherein such sugars or analogs are attached to a heterocyclic base (nucleic acid base) per se is known, and therefore need not be described here. Sugar modifications may also be made and combined with any phosphate modification in the preparation of a CpG-C ODN.

The heterocyclic bases, or nucleic acid bases, which are incorporated in the CpG-C ODN can be the naturally-occurring principal purine and pyrimidine bases, (namely uracil, thymine, cytosine, adenine and guanine, as mentioned above), as well as naturally-occurring and synthetic modifications of said principal bases. Thus, a CpG-C ODN may include one or more of inosine, 2′-deoxyuridine, and 2-amino-2′-deoxyadenosine.

According to another example the CPG-ODN is one of a Class A type CPG-ODNs (CPG-A ODNs), a Class B type CPG-ODNs (CPG-B ODNs), a Class P type CPG-ODNs (CPG-P ODN), and a Class S type CPG-ODNs (CPG-S ODN). In this regard, the CPG-A ODN can be CMP-001.

In another example, the CPG-ODN can be tilsotolimod (IMO-2125).

### Checkpoint Inhibitors

According to an example, the TLR agonists of the present application may be used in combination with a checkpoint inhibitor (CPI). The CPI can include a Programmed Death 1 receptor (PD-1) antagonist. A PD-1 antagonist can be any chemical compound or biological molecule that blocks binding of Programmed Cell Death 1 Ligand 1 (PD-L1) expressed on a cancer cell to PD-1 expressed on an immune cell (T cell, B cell or NKT cell) and preferably also blocks binding of Programmed Cell Death 1 Ligand 2 (PD-L2) expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and uses of the present application in which a human individual is being treated, the PD-1 antagonist blocks binding of human PD-L1 to human PD-1, and preferably blocks binding of both human PD-L1 and PD-L2 to human PD-1.

According to an example, the PD-1 antagonist can include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some examples the human constant region is selected from the group consisting of IgG1, IgG2, IgG3 and IgG4 constant regions, and in preferred examples, the human constant region is an IgG1 or IgG4 constant region. In some examples, the antigen binding fragment is selected from the group consisting of Fab, Fab′-SH, F(ab′)₂, scFv and Fv fragments.

According to an examples, the PD-1 antagonist can include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule.

According to an examples, the PD-1 antagonist can block PD-L1 expressed by tumor cells and MDSC, and other suppressive immune cells.

According to an examples, the PD-1 antagonist can inhibit the binding of PD-L1 to PD-1, and preferably also inhibits the binding of PD-L2 to PD-1. In some examples of the above treatment method, medicaments and uses, the PD-1 antagonist is a monoclonal antibody, or an antigen binding fragment thereof, which specifically binds to PD-1 or to PD-L1 and blocks the binding of PD-L1 to PD-1. In one example, the PD-1 antagonist is an anti-PD-1 antibody which comprises a heavy chain and a light chain.

According to an example, the PD-1 antagonist can be one of nivolumab, pembrolizumab, and cemiplimab. According to another example, nivolumab is administered intravenously (IV) via a peripheral vein at a dose of 480 mg every four weeks (“Q4W”) or 240 mg every two weeks (“Q2W”). According to another example, nivolumab is administered intravenously (IV) via a peripheral vein at a dose of nivolumab 360 mg every three weeks (“Q3W”). In another example, nivolumab dosing is weight-based, at nivolumab 3 mg/kg Q2W or 10 mg/kg Q2W. In another example, nivolumab dosing is weight-based at nivolumab 1 mg/kg Q3W. In yet another example, nivolumab is administered concomitantly, at the same time, at about the same time, or on the same day with SD-101. In another example, nivolumab is administered one a weekly, every other week, every three weeks, every four weeks, or on a monthly basis following the administration of one or more cycles of SD-101. A “cycle” of administration of SD-101 will be further described below.

According to another example, pembrolizumab is administered intravenously (IV) via a peripheral vein at a dose of 200 mg Q3W or 400 mg every 6 weeks (“Q6W”). In another example, pembrolizumab is administered concomitantly, at the same time, at about the same time, or on the same day with SD-101.

According to another example, the CPI can include a PD-L1 antagonist. In this regard, the PD-L1 antagonist can be one of atezolizumab, avelumab, and durvalumab.

According to another example, the CPI can include a CTLA-4 antagonist. In this regard, the CTLA-4 antagonist can be ipilimumab. According to another example, ipilimumab is administered intravenously (IV) via a peripheral vein at a dose of 3 mg/kg every three weeks. In yet another example, ipilimumab is administered concomitantly, at the same time, at about the same time, or on the same day with SD-101 and/or nivolumab. In another example, ipilimumab is administered once a week, every other week, every three weeks, every four weeks, or on a monthly basis following the administration of one or more cycles of SD-101 and/or nivolumab.

### Devices to Achieve Locoregional Delivery

According to an example, any of the TLR agonists, alone or in combination with a CPI, may be administered using any device useful to achieve locoregional delivery to a tumor, including a catheter itself, or may comprise a catheter along with other components (e.g., filter valve, balloon, pressure sensor system, pump system, syringe, outer delivery catheter, implantable port, etc.) that may be used in combination with the catheter. In certain examples, the catheter is a microcatheter.

In some examples, the device may have one or more attributes that include, but are not limited to, self-centering capability that can provide homogeneous distribution of therapy in downstream branching network of vessels; anti-reflux capability that can block or inhibit the retrograde flow of a pharmaceutical composition for infusion, e.g., a pharmaceutical fluid for infusion comprising a TLR agonist (for example, with the use of a valve and filter, and/or balloon); a system to measure the pressure inside the vessel; and a means or mechanism (e.g., a one-way valve that responds dynamically to local pressure changes, an intermittently occlusive valve and/or a porous balloon) to modulate the pressure inside the vessel, such as by causing a decrease in pressure at placement and during the TLR agonist infusion, and an increase of pressure during saline bolus or during bolus infusion of the TLR agonist. In some examples, the system is designed to continuously monitor real-time pressure or flow throughout the procedure. In one example, the mechanism for modulating the pressure generates, causes, and/or contributes to a net increase in fluid pressure within the vessel and/or target tissue or tumor. In one particular example, the mechanism for modulating the pressure may increase local vascular pressure at the target location, in particular, the increased pressure is greater than a base line arterial pressure. The mechanism for modulating the pressure may operate in sync with the cardiac cycle and/or facilitate antegrade flow. In some examples, the mechanism for modulating the pressure generates, causes, and/or contributes to a net decrease in fluid pressure within the vessel and/or target tissue or tumor. In some examples, the mechanism for modulating the pressure generates, causes, and/or contributes to first a decrease, then an increase in fluid pressure within the vessel and/or target tissue or tumor.

In some examples, the device that may be used to perform the methods of the present application is a device as disclosed in U.S. Pat. Nos. 8,500,775, 8,696,698, 8,696,699, 9,539,081, 9,808,332, 9,770,319, 9,968,740, 10,813,739, 10,588,636, 11,090,460, U.S. Patent Publication No. 2018/0193591, U.S. Patent Publication No. 2018/0250469, U.S. Patent Publication No. 2019/0298983, U.S. Patent Publication No. 2020/0038586, and U.S. Patent Publication No. 2020-0383688, which are all incorporated by reference herein in their entireties.

In some examples, the device is a device as disclosed in U.S. Pat. No. 9,770,319. In certain examples, the device may be a device known as the Surefire Infusion System.

In some examples, the device supports the measurement of intravascular pressure during use. In some examples, the device is a device as disclosed in U.S. Patent Publication No. 2020-0383688. In certain examples, the device may be a device known as the TriSalus Infusion System. In certain examples, the device may be a device known as the TriNav® Infusion System. The TriNav® is a single lumen catheter equipped with a one-way valve that responds dynamically to local pressure and flow changes, such as those arising from the cardiac cycle or generated by infusion. The valve structure modulates distal vascular pressures and blood flow. This in turn may alter therapeutic distribution and first-pass absorption due to increased contact time within the vasculature.

In some examples, the TLR agonist may be administered through a device via PEDD. In some examples, the TLR agonist may be administered while monitoring the pressure in the vessel, which can be used to adjust and correct the positioning of the device at the infusion site and/or to adjust the rate of infusion. Pressure may be monitored by, for example, a pressure sensor system comprising one or more pressure sensors.

The rate of infusion may be adjusted to alter vascular pressure or flow, which may promote the penetration and/or binding of the TLR agonist into the target tissue or tumor or at its surface. In some examples, the rate of infusion may be adjusted and/or controlled using a syringe pump as part of the delivery system or by any other method (e.g., an infusion flow rate regulating device). In some examples, the rate of infusion may be adjusted and/or controlled using a pump system. In some examples, the rate of infusion using a pump system may be about 0.1 cc/min to about 40 cc/min, or about 0.1 cc/min to about 30 cc/min, or about 0.5 cc/min to about 25 cc/min, or about 0.5 cc/min to about 20 cc/min, or about 1 cc/min to about 15 cc/min, or about 1 cc/min to about 10 cc/min, or about 1 cc/min to about 8 cc/min, or about 1 cc/min to about 5 cc/min. Further, the rate of infusion using a bolus infusion may be about 30 cc/min to about 360 cc/min, or about 120 cc/min to about 240 cc/min. In one example the SD-101 infusion procedure lasts approximately 10-200 minutes. In another example the SD-101 infusion procedure lasts approximately 10-60 minutes. In another example the SD-101 infusion procedure lasts approximately 25 minutes.

### Methods Comprising Administration to the Liver

In an example, the methods of the present application include methods of treating liver immune dysfunction, said method comprising administering a toll-like receptor (TLR) agonist to a patient in need thereof, wherein the TLR agonist is administered through a device by HAI to the liver. HAI refers to the infusion of a treatment into the hepatic artery of the liver or branches of the hepatic artery. According to an embodiment, the TLR agonist or agonists are introduced through the percutaneous introduction of a device into the branches of a hepatic artery or portal vein, such as a catheter and/or a device that facilitates pressure-enabled delivery. According to an example, the TLR agonist is a TLR9 agonist, more specifically, a class C TLR9 agonist, and in some examples, the class C TLR9 agonist is SD-101. In one example, the patient is a human patient.

According to another example, the methods of the present application include methods of treating liver immune dysfunction and tumor-induced immune dysfunction, said method comprising administering a toll-like receptor agonist to a patient in need thereof, wherein the toll-like receptor agonist is administered through a device by PVI to the liver. PVI refers to the infusion of a treatment into the hepatic portal venous system. According to an example, the toll-like receptor agonist or agonists are introduced through the percutaneous introduction of a device into the branches of the hepatic portal venous system, such as a catheter and/or a device that facilitates pressure-enabled delivery. According to an example, the toll-like receptor agonist is a TLR9 agonist, more specifically, a class C TLR9 agonist, and in some examples, the class C TLR9 agonist is SD-101. In one example, the patient is a human patient. According to another example, the methods include administration to a subject who is male or female, and is eighteen years of age or older.

According to another example, the methods of the present application can be administered with other cancer therapeutics such as immuno-modulators, tumor-killing agents, and/or other targeted therapeutics.

According to an example, TLR9 agonist therapy, specifically therapy with a class C TLR9 agonist, may be administered in combination with cell therapy (thereby enabling cell therapy by modulation of the immune system), chemoembolic treatment, or radioembolic treatment.

In one example, the above methods of administration to the liver are intended to result in the penetration of the toll-like receptor agonist, in particular, the TLR9 agonist, more particularly, the class C TLR9 agonist, and even more particularly, SD-101, throughout the solid tumor, throughout the entire organ, or substantially throughout the entire tumor. In an example, such methods enhance perfusion of the toll-like receptor agonist to a patient in need thereof, including by overcoming interstitial fluid pressure and solid stress of the tumor. In another example, perfusion throughout an entire organ or portion thereof, may provide benefits for the treatment of the disease by thoroughly exposing the tumor to therapeutic agent. In an example, such methods are better able to afford delivery of the toll-like receptor to areas of the tumor that have poor access to systemic circulation. In another example, such methods deliver higher concentrations of the toll-like receptor agonist into such a tumor with less toll-like receptor agonist delivered to nontarget tissues compared to conventional systemic delivery via a peripheral vein. Nontarget tissues are tissues directly perfused by the arterial network in immediate connection with the infusion device. In one example, such methods result in the reduction in size, reduction in growth rate, or shrinkage or elimination of the solid tumor.

The methods of the present application may also include mapping the vessels leading to the right and left lobes of the liver prior to performing HAI, or selective infusion into specific sectors or segments, and when necessary, occluding vessels that do not lead to the liver or that are otherwise not a target. In some examples, prior to infusion, patients can undergo a mapping angiogram, e.g. via a femoral artery approach.

Methods for mapping vessels in the body and delivery of therapeutics are well known to the ordinarily skilled artisan. Occlusion may be achieved, for example, through the use of microcoil embolization, which allows the practitioner to block off-target arteries or vessels, thereby optimizing delivery of the modified cells to the liver. Microcoil embolization can be performed as needed, such as prior to administering the first dose of TLR9 agonists, specifically class C TLR9 agonists, more specifically, SD-101 to facilitate optimal infusion of a pharmaceutical composition comprising the TLR9 agonists. In another example, a sterile sponge (e.g. GELFOAM) can be used. In this regard, the sterile sponge can be cut and pushed into the catheter. In another example, the sterile sponge can be provided as granules.

In some examples, doses of a TLR9 agonist, in particular, a class C TLR9 agonist, such as SD-101 may be about 0.01 mg, about 0.03 mg, about 0.05 mg, about 0.1 mg, about 0.3 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, or about 8 mg. In some examples, SD-101 is administered at doses of 12 mg, 16 mg, and 20 mg. Administration of a milligram amount of SD-101 (e.g. about 2 mg) describes administering about 2 mg of the composition illustrated in FIG. 1. For example, such an amount of SD-101 (e.g. about a 2 mg amount) may also exist within a composition that contains material in addition to such amount of SD-101, such as other related and unrelated compounds. Equivalent molar amounts of other pharmaceutically acceptable salts are also contemplated.

In some examples, doses of a TLR9 agonist, in particular, a class C TLR9 agonist, such as SD-101 may be between about 0.01 mg and about 20 mg, about 0.01 mg and about 10 mg, between about 0.01 mg and about 8 mg, and between about 0.01 mg and about 4 mg. In some examples, doses of a TLR9 agonist, in particular, a class C TLR9 agonist, such as SD-101 may be between about 2 mg and about 10 mg, between about 2 mg and about 8 mg, and between about 2 mg and about 4 mg. In some examples, doses of a TLR9 agonist, in particular, a class C TLR9 agonist, such as SD-101 may be less than about 10 mg, less than about 8 mg, less than about 4 mg, or less than about 2 mg. Such doses may be administered daily, weekly, or every other week. In one example, doses of SD-101 are incrementally increased, such as through administration of about 2 mg, followed by about 4 mg, and then followed by about 8 mg.

In some examples, the methods of the present application may comprise administering a dosing regimen comprising cycles, in which one or more of the cycles comprise administering SD-101 via HAI and/or PEDD. As used herein, a “cycle” is a repeat of a dosing sequence. In one example, one cycle comprises three weekly doses per cycle (i.e. administration of SD-101 once per week over three consecutive weeks). In one example, a cycle of treatment according to the present application may comprise periods of SD-101 administration followed by “off” periods or rest periods. In another example, in addition to three weekly doses per cycle, the cycle further comprises one week, two weeks, three weeks, or four weeks as a rest period following the weekly administration of SD-101. In yet another example, in addition to three weekly doses per cycle, the cycle further comprises about thirty-eight days as a rest period following the weekly administration of SD-101. In another example, the entire cycle comprises about fifty-two days. In another example, the dosing regimen comprises at least one, at least two, or at least three cycles, or longer.

In some examples, the present application relates to the use of a TLR9 agonist, in particular, a class C TLR9 agonist in the manufacture of a medicament for treating liver immune dysfunction and tumor-induced immune dysfunction, said method comprising administering the TLR9 agonist, more particularly, the class C TLR9 agonist to a patient in need thereof, wherein the TLR9 agonist, more specifically, the class C TLR9 agonist is administered through a device by HAI to the liver.

In some examples, SD-101 is administered for the treatment of liver immune dysfunction at a dose of 2 mg through HAI, and in some examples, the SD-101 is further administered through a device that modulates pressure (i.e. PEDD). In some examples, SD-101 is administered at a dose of 2 mg through HAI through a device that modulates vascular pressure in combination with a CPI, wherein the CPI is nivolumab. In other examples, SD-101 is administered at a dose of 2 mg through HAI and through a device that modulates pressure in combination with ipilimumab. In some examples, SD-101 is administered at a dose of 2 mg through HAI and through a device that modulates pressure in combination with ipilimumab and nivolumab. In other examples, SD-101 is administered at a dose of 2 mg through HAI and through a device that modulates pressure in combination with pembrolizumab.

In some examples, SD-101 is administered for the treatment of liver immune dysfunction at a dose of 4 mg through HAI, and in some examples, the SD-101 is further administered through a device that modulates pressure (e.g., dynamically responding to local pressure changes, more specifically, PEDD). In some examples, SD-101 is administered at a dose of 4 mg through HAI through a device that modulates vascular pressure (e.g., dynamically responding to local pressure changes, more specifically, PEDD) in combination with a CPI. The CPI may be administered systemically or locoregionally. For example, the CPI may be administered intravenously (IV), subcutaneously (SQ), intraperitoneally (IP), or through HAI, such as, for example, through a device that modulates pressure (e.g., dynamically responding to local pressure changes, more specifically, PEDD).

In some examples, SD-101 is administered at a dose of 4 mg through HAI through a device that modulates vascular pressure (e.g., dynamically responding to local pressure changes, more specifically, PEDD) in combination with a CPI, administered systemically (e.g., IV or SQ) or locoregionally (e.g., IP or through HAI, such as via PEDD), wherein the CPI is nivolumab. In other examples, SD-101 is administered at a dose of 4 mg through HAI and through a device that modulates pressure (e.g., dynamically responding to local pressure changes, more specifically, PEDD) in combination with ipilimumab, administered systemically (e.g., IV or SQ) or locoregionally (e.g., IP or through HAI, such as via PEDD). In some examples, SD-101 is administered at a dose of 4 mg through HAI and through a device that modulates pressure (e.g., dynamically responding to local pressure changes, more specifically, PEDD) in combination with ipilimumab and nivolumab, administered systemically (e.g., IV or SQ) or locoregionally (e.g., IP or through HAI, such as via PEDD). In other examples, SD-101 is administered at a dose of 4 mg through HAI and through a device that modulates pressure (e.g., dynamically responding to local pressure changes, more specifically, PEDD) in combination with pembrolizumab, administered systemically (e.g., IV or SQ) or locoregionally (e.g., IP or through HAI, such as via PEDD).

In some examples, SD-101 is administered for the treatment of liver immune dysfunction at a dose of 8 mg through HAI, and in some embodiments, the SD-101 is further administered through a device that modulates pressure (e.g., dynamically responding to local pressure changes, more specifically, PEDD). In some examples, SD-101 is administered at a dose of 8 mg through HAI through a device that modulates vascular pressure (e.g., dynamically responding to local pressure changes, more specifically, PEDD) in combination with a CPI, wherein the CPI is nivolumab, administered systemically (e.g., IV or SQ) or locoregionally (e.g., IP or through HAI, such as via PEDD). In other examples, SD-101 is administered at a dose of 8 mg through HAI and through a device that modulates pressure (e.g., dynamically responding to local pressure changes, more specifically, PEDD) in combination with ipilimumab, administered systemically (e.g., IV or SQ) or locoregionally (e.g., IP or through HAI, such as via PEDD). In some examples, SD-101 is administered at a dose of 8 mg through HAI and through a device that modulates pressure (e.g., dynamically responding to local pressure changes, more specifically, PEDD) in combination with ipilimumab and nivolumab, administered systemically (e.g., IV or SQ) or locoregionally (e.g., IP or through HAI, such as via PEDD). In other examples, SD-101 is administered at a dose of 8 mg through HAI and through a device that modulates pressure (e.g., dynamically responding to local pressure changes, more specifically, PEDD) in combination with pembrolizumab, administered systemically (e.g., IV or SQ) or locoregionally (e.g., IP or through HAI, such as via PEDD).

In some examples, the methods of the present application result in the treatment of target lesions, e.g., target liver cancer lesions or tumors. In this example, the methods of the present application may result in a complete response, comprising the disappearance of all target lesions, e.g., target liver cancer lesions or tumors. In some examples, the methods of the present application may result in a partial response, comprising at least a 30% decrease in the sum of the longest diameter of target lesions, e.g., target liver cancer lesions or tumors, taking as reference the baseline sum longest diameter. In some examples, the methods of the present application may result in stable disease of target lesions, e.g., target liver cancer lesions or tumors, comprising neither sufficient shrinkage to qualify for partial response nor sufficient increase to qualify for progressive disease, taking as reference the smallest sum longest diameter since the treatment started. In such an example, progressive disease is characterized by at least a 20% increase in the sum of the longest diameter of target lesions, e.g., target liver cancer lesions or tumors, taking as reference the smallest sum longest diameter recorded since the treatment started or the appearance of one or more new lesions. The sum must demonstrate an absolute increase of or about 5 mm. In some examples, response in the form of decrease in size may be offset by tumor killing and inflammation resulting in swelling of the tumor (known as pseudoprogression).

In another example, the methods of the present application result in the treatment of nontarget lesions. Nontarget lesions are lesions not directly perfused by the arterial network in immediate communication with the infusion system. In this example, the methods of the present application may result in a complete response, comprising the disappearance of all nontarget lesions or elimination of all viable tumor. In some examples, the methods of the present application result in persistence of one or more nontarget lesion(s), while not resulting in a complete response or progressive disease. In such an example, progressive disease is characterized by unequivocal progression of existing nontarget lesions, and/or the appearance of one or more new lesions.

In some examples, the methods of the present application result in an increased duration of overall response. In some examples, the duration of overall response is measured from the time measurement criteria are met for complete response or partial response (whichever is first recorded) until the first date of recurrent or progressive disease, for example the first date that recurrent or progressive disease is objectively documented (taking as reference for progressive disease the smallest measurements recorded since the treatment started). The duration of overall complete response may be measured from the time measurement criteria are first met for complete response until the first date of recurrent or progressive disease, for example, the first date that progressive disease is objectively documented. In some examples, the duration of stable disease is measured from the start of the treatment until progressive disease is observed, for example, the criteria for progression are met, taking as reference the smallest measurements recorded since the treatment started, including the baseline measurements.

In yet other examples, the methods of the present application result in improved overall survival rates. For example, overall survival may be calculated from the date of initiating treatment until the time of death. In a particular example, in a clinical trial of the treatment, the date of enrollment to the time of death. Patients who are still alive prior to the data cutoff for final efficacy analysis in a clinical trial study, or who dropout prior to study end, will be censored at the day they were last known to be alive.

In other examples, the methods of the prevent application result in progression-free survival. Progression-free survival may be determined from the date of initiating treatment until relapse or death, whichever occurs first. In a particular example, for instance, progression-free survival may be calculated from the date of enrollment in a clinical trial of the treatment to the time of CT scan documenting relapse (or other unambiguous indicator of disease development), or date of death, whichever occurs first. Patients who have no documented relapse and are still alive prior to the data cutoff for final efficacy analysis, or who drop out prior to study end, will be censored at the date of the last radiological evidence documenting absence of relapse.

According to another example, the methods of the present application include a method for treating liver immune dysfunction. Liver immune dysfunction refers to immune dysfunction in the liver tumor microenvironment (TME) reducing the ability of the patient's immune system to attack cancer cells and/or the ability of immunotherapy to induce a beneficial therapeutic response. Liver immune dysfunction may be characterized by a number of different biologic characteristics, such as, for example, an increased level of MDSCs present in the liver TME, a decreased level of T-cells and/or macrophages (e.g., M1 and/or M2 macrophages) present in the liver TME, a decreased level of mature dendritic cells, reduced immune control of the liver cancer, and/or reduced responsiveness to anti-PD-1 therapy. Other biologic characteristics of liver immune dysfunction include: activation of immune cells in the normal liver (non-tumor liver in the same patient), with migration into liver metastases; activation and mobilization of peripheral blood immune cells (T, NK, B, cytotoxic T, CD4 Th1 T, exhausted T, macrophages); increased TLR signaling; increased leukocytes (CD45+); increased exhausted CD8 T cells (which is very important for checkpoint responsiveness); induction of Th1 programming; reduction of Th2 programming; increased T cell receptor and T cell co-stimulatory signaling; increased IL9, IL15, CCL7; B cell activation; induction of mast cells; induction of NK cells; induction of IFNg and related genes; increased interferon signaling; increased chemokine signaling; increased IL6 in the liver without an increase in the blood; increased M1/activated macrophage genes: CD68, CD86 dendritic cell activation and migration to draining lymph nodes; decreases in MDSC associated genes: IDO1, TOD2, ARG-1, NOS2, TIMD4; decreased angiogenesis and VEGF; decreased fatty acid oxidation (a preferred MDSC program); and/or increase in PD-L1, IFNγ and IP10 gene expression.

In one example, the methods of the present application include a method for treating liver immune dysfunction, wherein the administration of SD-101 results in a reduction of tumor burden. The tumor burden may be determined using any suitable method, for example, a length of a longest diameter of a tumor lesion or a volume of the tumor lesion. In some examples, the tumor burden is reduced by about 10%, by about 20%, by about 30%, by about 40%, by about 50%, by about 60%, by about 70%, by about 80%, by about 90%, or by about 100%. In one example, the tumor burden is reduced by about 10%, by about 20%, by about 30%, by about 40%, by about 50%, by about 60%, by about 70%, by about 80%, by about 90%, or by about 100%, as determined by the length of the longest diameter of the tumor lesions. In another example, the tumor is reduced by about 10%, by about 20%, by about 30%, by about 40%, by about 50%, by about 60%, by about 70%, by about 80%, by about 90%, or by about 100%, as determined by volume of the tumor lesion.

According to another example, the methods of the present application include a method for treating liver immune dysfunction, wherein the administration of SD-101 results in a reduction of tumor progression or stabilization of tumor growth. In some examples, tumor progression is reduced by about 10%, by about 20%, by about 30%, by about 40%, by about 50%, by about 60%, by about 70%, by about 80%, by about 90%, or by about 100%. The tumor burden may be determined using any suitable method, for example, a length of a longest diameter of a tumor lesion or a volume of the tumor lesion. In one example, tumor progression is reduced by about 10%, by about 20%, by about 30%, by about 40%, by about 50%, by about 60%, by about 70%, by about 80%, by about 90%, or by about 100%, as determined by the length of the longest diameter of the tumor lesions. In another example, tumor progression is reduced by about 10%, by about 20%, by about 30%, by about 40%, by about 50%, by about 60%, by about 70%, by about 80%, by about 90%, or by about 100%, as determined by the length of the longest diameter of the tumor lesions, as determined by volume of the tumor lesion.

According to another example, the methods of the present application include a method for treating liver immune dysfunction, wherein the administration of SD-101 decreases MDSC in the liver TME, enables increased immune control of the liver cancer and/or improves responsiveness to systemic anti-PD-1 therapy. For example, the method for treating liver immune dysfunction includes administration of SD-101 to reprogram the liver MDSC compartment to enable immune control of the liver cancer and/or improve responsiveness to systemic anti-PD-1 therapy through elimination of MDSC from the liver TME. In some examples, the methods of the present application are superior in controlling amount of MDSC in the liver TME. In some examples, the methods of the present application include a method for treating liver immune dysfunction, wherein the administration of SD-101 reduces the frequency of MDSC cells (e.g., CD11b+Gr1+), monocytic MDSC (M-MDSC; e.g., CD11b+Ly6C+) cells, granulocytic MDSC (G-MDSC; e.g., CD11b+LY6G+) cells, and/or human MDSC (e.g., CD33+CD11b+HLADR− (e.g., CD14+ for m-MDSC and CD15+ for G-MDSC)) in the liver TME. According to another example, the methods of the present application enhance amount of M1 macrophages (e.g., CD14+CD86+) in the liver TME. According to yet another example, the methods of the present application decrease M2 macrophages (e.g., CD14+CD163+CD206+) in the liver TME. According to yet another example, the methods of the present application increase monocytic dendritic cells (e.g., CD14⁺CD11c⁺CD123⁻) in the liver TME. According to an example, the MDSC, M1, M2 and dendritic cell effects as described in this paragraph apply to human phenotypes including but not limited to: human M-MDSC: (e.g., CD33+CD11b+HLA-DR-CD14+); human G-MDSC: (e.g., CD33+CD11b+HLA-DR-CD15+); human M1 macrophages: (e.g., CD14+CD86+); human M2 macrophages: (e.g., CD14+CD163+CD206+); and human monocytic dendritic cells (e.g., CD14⁺CD11c⁺CD123⁻).

In another example, the methods of the present application increase NFκB activation and other molecules related to TLR9 signaling, for example, in the liver TME. In yet an additional example, the methods of present application increase IL-6, for example, in the liver TME. In another example, the methods of the present application increase IL10, for example, in the liver TME. In yet an additional example, the methods of present application increase IL-29, for example, in the liver TME. In another embodiment, the methods of the present application increase IFNα, for example, in the liver TME. As a further embodiment, the methods of the present application decrease STAT3 phosphorylation, for example, in the liver TME.

In another example, the methods of the present application include a method for treating liver immune dysfunction, wherein administration of a TLR9 agonist, specifically, a class C TLR9 agonist, and more specifically, SD-101, results in at least one of the following changes in gene expression within liver metastases:
activation of immune cells in the normal liver (non-tumor liver in the same patient), with migration into liver metastases;
activation and mobilization of peripheral blood immune cells (T, NK, B, cytotoxic T, CD4 Th1 T, exhausted T, macrophages);
increased TLR signaling; increased leukocytes (CD45+);
increased exhausted CD8 T cells (which is very important for checkpoint responsiveness);
induction of Th1 programming;
reduction of Th2 programming;
increased T cell receptor and T cell co-stimulatory signaling;
increased IL9, IL15, CCL7;
B cell activation;
induction of mast cells;
induction of NK cells;
induction of IFNg and related genes;
increased interferon signaling;
increased chemokine signaling;
increased IL6 in the liver without an increase in the blood;
increased M1/activated macrophage genes: CD68, CD86 dendritic cell activation and migration to draining lymph nodes;
decreases in MDSC associated genes: IDO1, TOD2, ARG-1, NOS2, TIMD4;
decreased angiogenesis and VEGF;
decreased fatty acid oxidation (a preferred MDSC program); and/or
increase in PD-L1, IFNγ and IP10 gene expression.

In another embodiment, the methods of the present application include a method for administering a TLR 9 agonist, specifically, a class C TLR9 agonist, in particular, SD-101, in combination with a checkpoint inhibitor to a patient, wherein the administration of the TLR9 agonist, specifically, the class C TLR9 agonist, and more specifically SD-101 in combination with the CPI results in a decrease in circulating tumor cell levels and/or circulating tumor DNA levels, thereby resulting in a decrease of the tumor burden. In one example, the CPI may be administered systemically (e.g., IV or SQ) or locoregionally (e.g., IP or through HAI, such as via PEDD).

The present application will be further illustrated and/or demonstrated in the following Examples, which is given for illustration/demonstration purposes only and is not intended to limit the invention in anyway.

### EXAMPLES

### Example 1

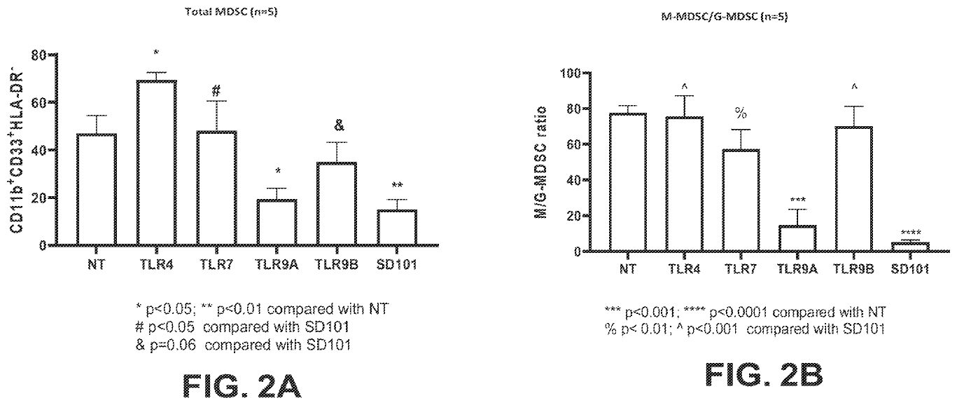

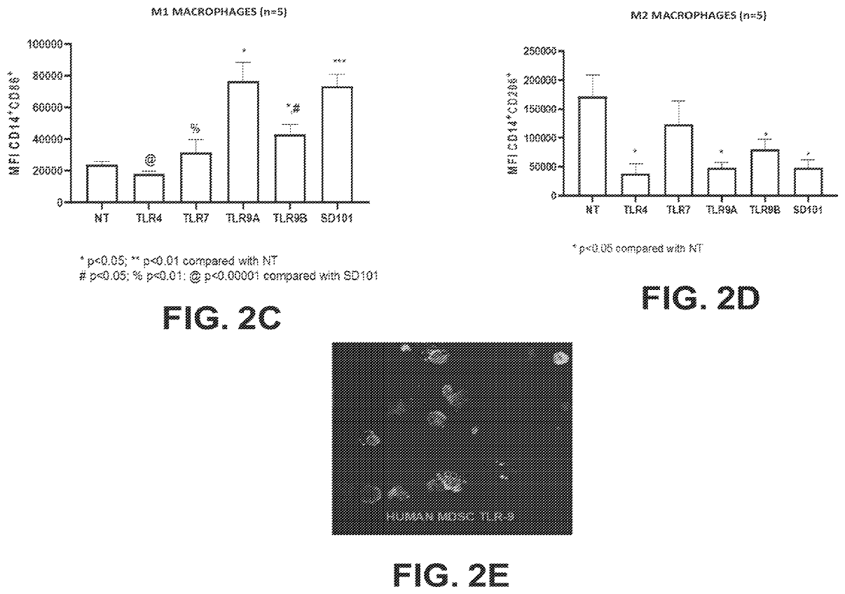

In Example 1, Peripheral Blood Mononuclear Cells (PBMCs) obtained from human donors were cultured in vitro in the presence of or in the absence of IL6 and GMCSF. The presence of IL6 and GMCSF is used to induce MDSCs. The treated cells were then evaluated using flow cytometry to determine the effect of different TLR agonists on MDSCs (M-MDSC and G-MDSC) and macrophages (M1 and M2 macrophages). The data generated by flow cytometry are shown in FIGS. 2A-2D.

FIGS. 2A-2E illustrate an in vitro analysis of human PMBCs harvested from healthy donors and treated with IL6 and GM-CSF to induce MDSC, according to Example 1.

In particular, FIG. 2A shows amount of total MDSC induced in the in vitro samples (i.e., NT (not treated) or treated with a TLR4 agonist, TLR7 agonist, TLR7 agonist, TLR9A agonist, TLR9B agonist or SD101). The data of FIG. 2A demonstrates the effect of a TLR4 agonist, a TLR7 agonist, a TLR9A agonist, a TLR9B agonist and SD-101 as compared to non-treated control on amount of total MDSCs induced by treatment of human PBMCs with IL6 and GMCSF. In FIG. 2A, * indicates p<0.05 compared with NT, ** indicates p<0.01 compared with NT, # indicates p<0.05 compared with SD-101, & indicates p=0.006 compared with SD-101. FIG. 2B demonstrates the effect of the TLR4 agonist, the TLR7 agonist, the TLR9A agonist, the TLR9B agonist and SD-101 as compared to non-treated control on amount of monocytic MDSC (M-MDSC) and granulocytic MDSC (G-MDSC) induced by treatment of human PBMCs with IL6 and GMCSF. In FIG. 2B. *** indicates p<0.001 compared with NT, **** indicates p <0.0001 compared with NT, % indicates p<0.01 compared with SD-101, and \hat{ } indicatges p<0.001 compared with SD-101. As can be seen in FIGS. 2A and 2B, SD-101 demonstrated (i) better elimination of the dominant MDSC subset in the liver, e.g., M-MDSC and (ii) a more favorable effect on the M1/M2 macrophage ratio (i.e., induction of M1 and reduction of M2). FIGS. 2C and 2D demonstrate the effect of the TLR4 agonist, the TLR7 agonist, the TLR9A agonist, the TLR9B agonist and SD-101 as compared to non-treated control on amounts of M1-macrophages and M2 macrophages induced by treatment of human PBMCs with IL6 and GMCSF, respectively. In FIG. 2C, * indicates p<0.05 compared with NT, ** indicates p<0.01 compared with NT, # indicates p<0.05 compared with SD-101, @ indicates p<0.00001 compared with SD-101. In FIG. 2D, * indicates p<0.05 compared with NT. For each of the data sets shown in FIGS. 2A-2D, the number of different human donors is n=5.

FIG. 2E shows the an image of MDSC cells induced from PMBCs when the cells are treated with a TLR9 agonist.

In view of the data shown in FIGS. 2A-2E, these in vitro studies of human PMBCs treated with IL6 and GM-CSF to induce MDSC demonstrate that SD-101 is superior to class B TLR9 agonist and a TLR7 agonist with respect to reduction of MDSC, reduction of M2 macrophages, and promotion of M1 macrophages.

### Example 2

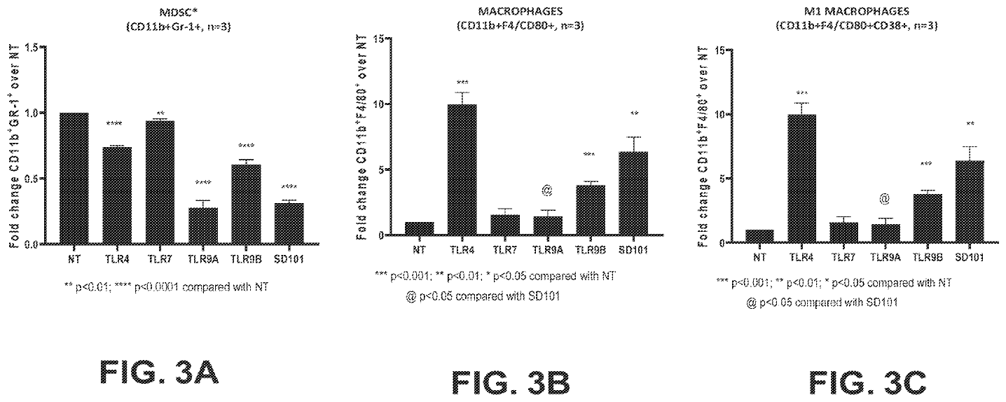

In Example 2, murine bone marrow cells from mice with or without tumor were cultured in vitro in the presence of or in the absence GMCSF. The presence GMCSF is used to induce MDSCs. The treated cells were then evaluated using flow cytometry to determine the effect of different TLR agonists on total MDSCs, total macrophages, and M1 macrophages in this murine model. The data generated by flow cytometry are shown in FIGS. 3A-3C.

FIG. 3A demonstrates the effect of a TLR4 agonist, a TLR7 agonist, a TLR9A agonist, a TLR9B agonist and SD-101 as compared to non-treated control on amount of MDSC (CD11b+Gr-1+) induced by treatment of murine bone marrow cells with GMCSF. In FIG. 3A, ** indicates p<0.01 compared with NT and **** indicates p<0.0001 compared with NT. FIG. 3B demonstrates the effect of a TLR4 agonist, a TLR7 agonist, a TLR9A agonist, a TLR9B agonist and SD-101 as compared to non-treated control on amount of macrophages (CD11b+F4/CD80+) induced by treatment of murine bone marrow cells with GMCSF. In FIG. 3B, *** indicates p<0.001 compared with NT, * indicates p<0.05 compared with NT, and @ indicates p<0.05 compared with SD-101. FIG. 3C demonstrates the effect of a TLR4 agonist, a TLR7 agonist, a TLR9A agonist, a TLR9B agonist and SD-101 as compared to non-treated control on amount of M1 macrophages (CD11b+F4/CD80+CD38+) induced by treatment of murine bone marrow cells with GMCSF. In FIG. 3C, *** indicates p<0.001 compared with NT, ** indicates p<0.01 compared with NT, * indicates p<0.05 compared with NT, and @ indicates p<0.05 compared with SD-101. For each of the data sets shown in FIGS. 3A-3C, the number of different human donors is n=3.

As can be seen in the data of FIGS. 3A-3C, in vitro studies of murine bone marrow cells demonstrate that SD-101 is similarly superior to a class B TLR9 agonist and a TLR7 agonist for murine myeloid cell programming. In particular, FIGS. 3A-3C depict in vitro analysis of murine bone marrow cells, in which GM-CSF was used to induce MDSC, from mice with or without tumor. In this regard, SD-101 was superior to class A TLR9, class B TLR9, and TLR7 agonists in reduction of MDSC and M2 macrophages, in addition to induction of M1 macrophages.

### Example 3

In Example 3, infusions of a class C TLR9 agonist, SD-101, were administered via hepatic artery infusion (PEDD/HAI) with the aim of enhancing response rates to CPI therapy in patients with uveal melanoma liver metastases. Four patients were enrolled and treated with a dose of 2 mg SD-101 for each of three weekly SD-101 infusions via HAI and PEDD in a first cycle. Pharmacokinetic (PK) data collected in this study suggest that the PEDD/HAI delivery approach is achieving its goal of obtaining high concentrations of SD-101 in liver tissue, and transient and low levels of systemic exposure to SD-101, as determined based on plasma levels (<2 hours, <150 ng/ml). Cell-mediated anti-tumor immunity (Th1, TCR/co-stimulation, CD8 T cells, cytotoxic cells, B cells, NK cells) increased in uveal melanoma liver metastases. A simultaneous decrease in normal liver tissue suggested a shift into an adjacent tumor, showing the importance of treating the entire immunosuppressive organ. CD8 T, Th1, B, and NK increased in peripheral blood at later time points with serum IFNγ induction. As for cytokine programming, IFNγ related genes increase in liver metastases and serum increases, and Th1 gene increased in liver metastases with a decrease in Th2. MDSC associated genes decreased (NOS-2, ARG-1, IDO-1, TDO-2). Changes in the liver were seen four weeks after three infusions of 2 mg SD-101 via PEDD.

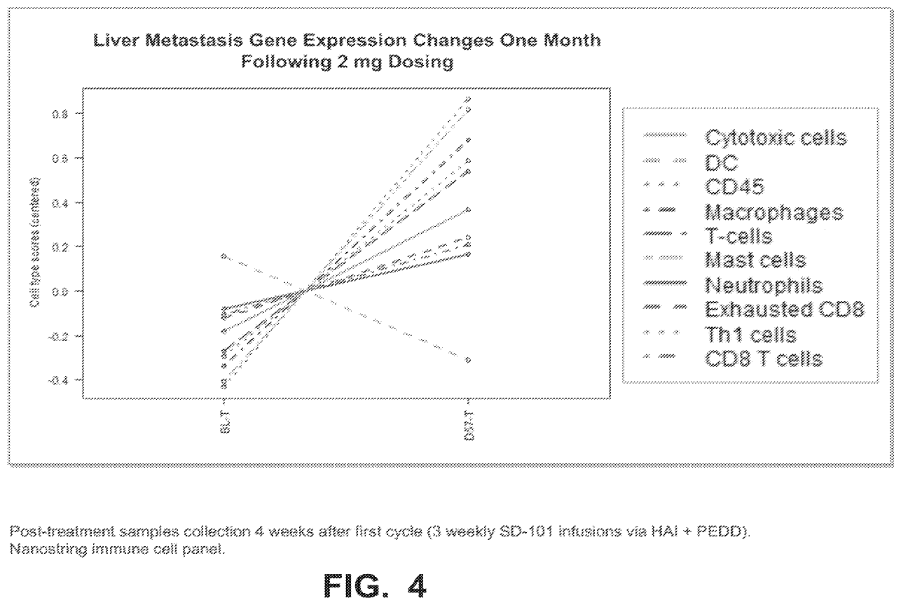

Post-treatment liver metastasis samples (collected four weeks after the first cycle of three weekly SD-101 infusions via HAI and PEDD) from these patients were tested in a NanoString® immune cell panel to profile the expression of gene signatures for immune cells, including cytotoxic cells, dendritic cells (DC), CD45, macrophages, T-cells, mast cells, neutrophils, exhausted CD8, Th1 cell, and CD8 T cells. Data from the NanoString® immune cell panel is shown in FIG. 4 where BL-T refers to baseline time and D57-T refers to a time 4 weeks after the first cycle of 3 weekly infusions. As illustrated in FIG. 4, a NanoString® immune cell panel of post-treatment liver metastasis samples (collected four weeks after the first cycle of three weekly SD-101 infusions via HAI and PEDD) showed, among other things, increases in gene signatures including cytotoxic cells, macrophages, T cells, and exhausted CD8 T cells, and decreases in gene signatures including dendritic cells, possibly due to migration to draining lymph nodes. In this regard, the liver metastasis samples were taken from the four patients (average relative gene expression levels from four pre-treatment and two post-treatment).

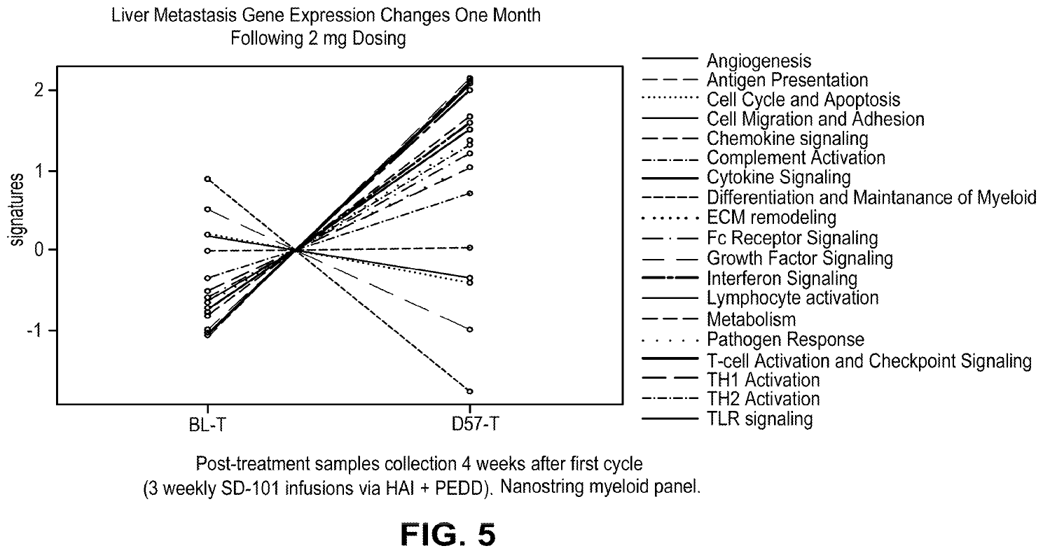

Post-treatment liver metastasis samples (collected four weeks after the first cycle of three weekly SD-101 infusions via HAI and PEDD) from these patients were tested in a NanoString® myeloid panel to profile the expression of myeloid gene signatures, including angiogenesis, antigen presentation, cell cycle and apoptosis, cell migration and adhesion, chemokine signaling, complement activation, cytokine signaling, differentiation and maintenance of myeloid, ECM remodeling, Fc receptor signaling, growth factor signaling, interferon signaling, lymphocyte activation, metabolism, pathogen response, T-cell activation and checkpoint signaling, Th1 activation, Th2 activation, and TLR signaling. Data from the NanoString® myeloid panel is shown in FIG. 5 where BL-T refers to baseline time and D57-T refers to a time 4 weeks after the first cycle of 3 weekly infusions. As illustrated in FIG. 5, a NanoString® myeloid panel of post-treatment liver metastasis samples (collected four weeks after the first cycle of three weekly SD-101 infusions via HAI and PEDD) showed, among other things, increases in gene signatures including lymphocyte activation, Th1 activation, interferon and TLR signaling, antigen presentation, exhausted CD8 T cells, and decreases in gene signatures including Th2 activation and angiogenesis. In this regard, the liver metastasis samples were taken from the four patients (average relative gene expression levels from four pre-treatment and two post-treatment).

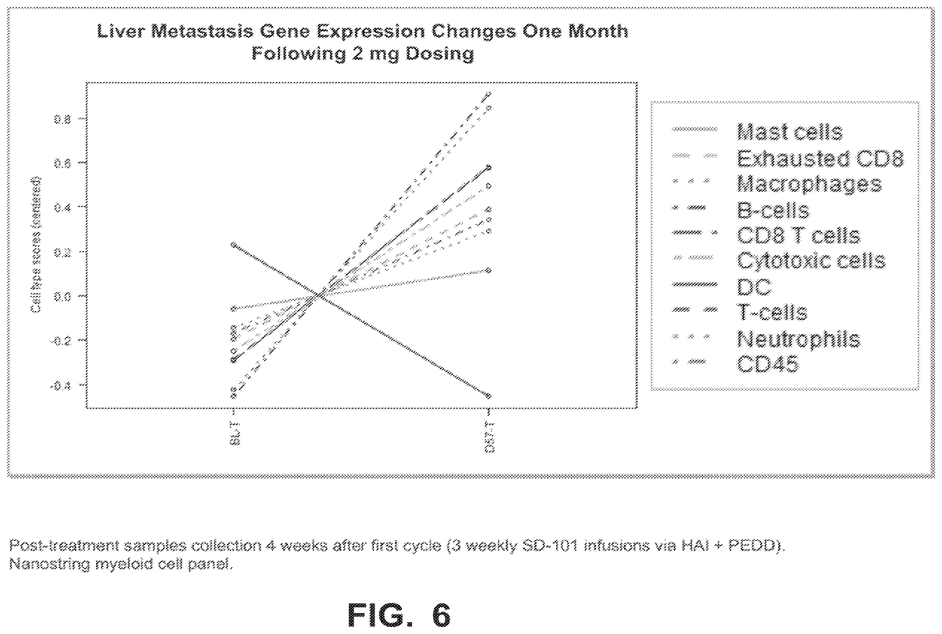

Post-treatment liver metastasis samples (collected four weeks after the first cycle of three weekly SD-101 infusions via HAI and PEDD) from these patients were tested in a NanoString® myeloid cell panel to profile the expression of gene signatures for myeloid cells, including mast cells, exhausted CD8, macrophages, B-cells, CD8 T cells, DC, T-cells, neutrophils, and CD45. Data from the NanoString® myeloid cell panel is shown in FIG. 6 where BL-T refers to baseline time and D57-T refers to a time 4 weeks after the first cycle of 3 weekly infusions. As illustrated in FIG. 6, a NanoString® myeloid cell panel of post-treatment liver metastasis samples (collected four weeks after the first cycle of three weekly SD-101 infusions via HAI and PEDD) showed, among other things, increases in gene signatures including B cells, CD8 T cells, cytotoxic cells, and macrophages, and decreases in gene signatures including dendritic cells, possibly due to migration to draining lymph nodes. In this regard, the liver metastasis samples were taken from the four patients (average relative gene expression levels from four pre-treatment and two post-treatment).

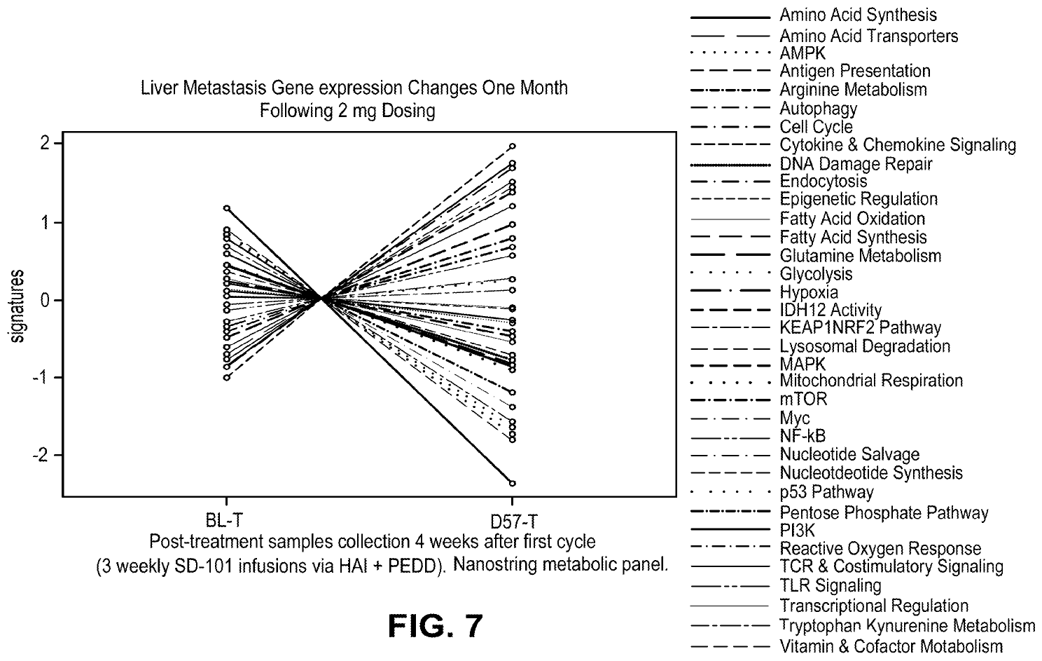

Post-treatment liver metastasis samples (collected four weeks after the first cycle of three weekly SD-101 infusions via HAI and PEDD) from these patients were tested in a NanoString® metabolic panel to profile the expression of metabolic gene signatures, including amino acid transporters, AMPK, antigen presentation, arginine metabolism, autophagy, cell cycle, cytokine & chemokine signaling, DNA damage repair, endocytosis, epigenetic regulation, fatty acid oxidation, fatty acid synthesis, glutamine metabolism, glycolysis, hypoxia IDH12 activity, KEAP1NBRF2 pathway, Lysosomal degradation, MAPK, mitochondrial respiration, mTOR, Myc, NF-κB, nucleotide salvage, nucleotide synthesis, p53 pathway, pentose phosphate pathway, P13K, reactive oxygen response, TCR & costimulatory signaling, TLR signaling, transcriptional regulation, tryptophanKynurenine metabolism, and Vitamin & Cofactor metabolism. Data from the NanoString® metabolic panel is shown in FIG. 7 where BL-T refers to baseline time and D57-T refers to a time 4 weeks after the first cycle of 3 weekly infusions. As illustrated in FIG. 7, a NanoString® metabolic panel of post-treatment liver metastasis samples (collected four weeks after the first cycle of three weekly SD-101 infusions via HAI and PEDD) showed, among other things, increases in gene signatures including antigen presentation, chemokine and cytokine signaling, and TCR and costimulatory signaling, decreases in gene signatures including cell cycle and fatty acid oxidation, and decreases in MDSC related genes including ARG1, NOS2, and GM-CSF-R. In this regard, the liver metastasis samples were taken from the four patients (average relative gene expression levels from four pre-treatment and two post-treatment).

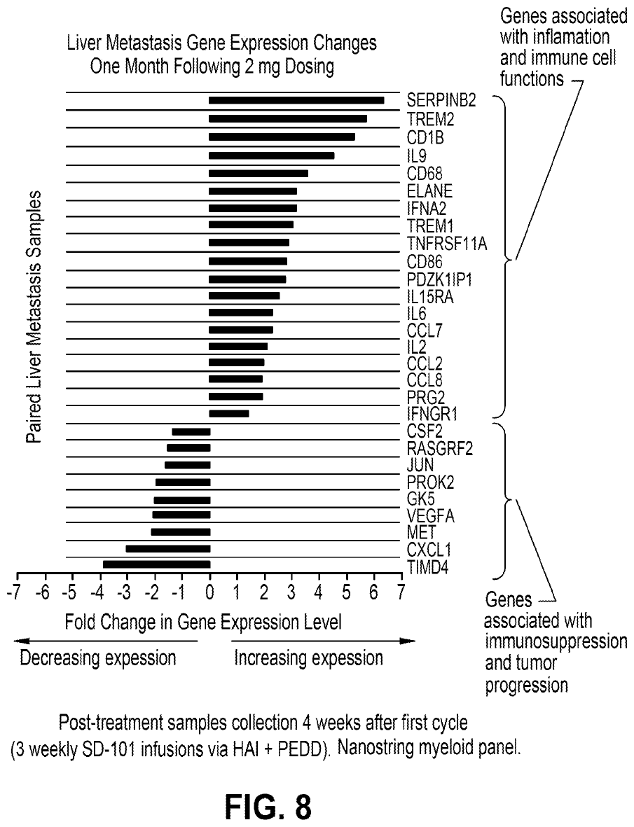

Post-treatment liver metastasis samples (collected four weeks after the first cycle of three weekly SD-101 infusions via HAI and PEDD) from these patients were tested in a NanoString® myeloid panel to profile the expression of myeloid gene signatures, including genes associated with inflammation and immune cell functions and genes associated with immunosuppression and tumor progression. Data from the NanoString® myeloid panel is shown in FIG. 8. As illustrated in FIG. 8, a NanoString® myeloid panel of post-treatment liver metastasis samples (collected four weeks after the first cycle of three weekly SD-101 infusions via HAI and PEDD) showed increased expression of genes associated with inflammation and immune cell functions (e.g., SERPINB2, TREM2, CD18, IL9, CD68, ELANE, IFNA2, TREM1, TNFRSF11A, CD86, PDZK1IP1, IL15RA, IL6, CCL7, IL2, CCL2, CCL8, PRG2, and IFNGR1), and decreased expression of genes associated with immunosuppression and tumor progression (e.g., CSF2, RSGRF2, JUN, PROk2, GKS, VEGA, MET, CXCL1, TIMD4). In particular, the figure illustrates increases in gene signatures including TREM2 (associated with TMB and MSI), IFNA2 and IFNGR1, CD68, CD86, IL2, IL9, IL15, and CCL7, and decreases in gene signatures including CSF2 (GM-CSF), IDO-1, TDO-2, VEGFA, and TIMD4 (role in MDSC suppressive function). In this regard, the liver metastasis samples were taken from the four patients (average relative gene expression levels from four pre-treatment and two post-treatment).

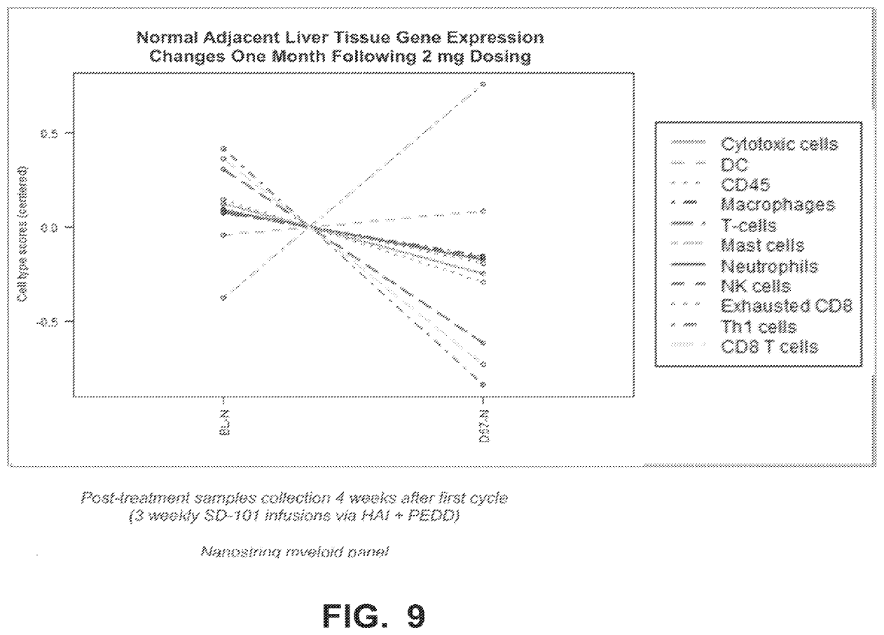

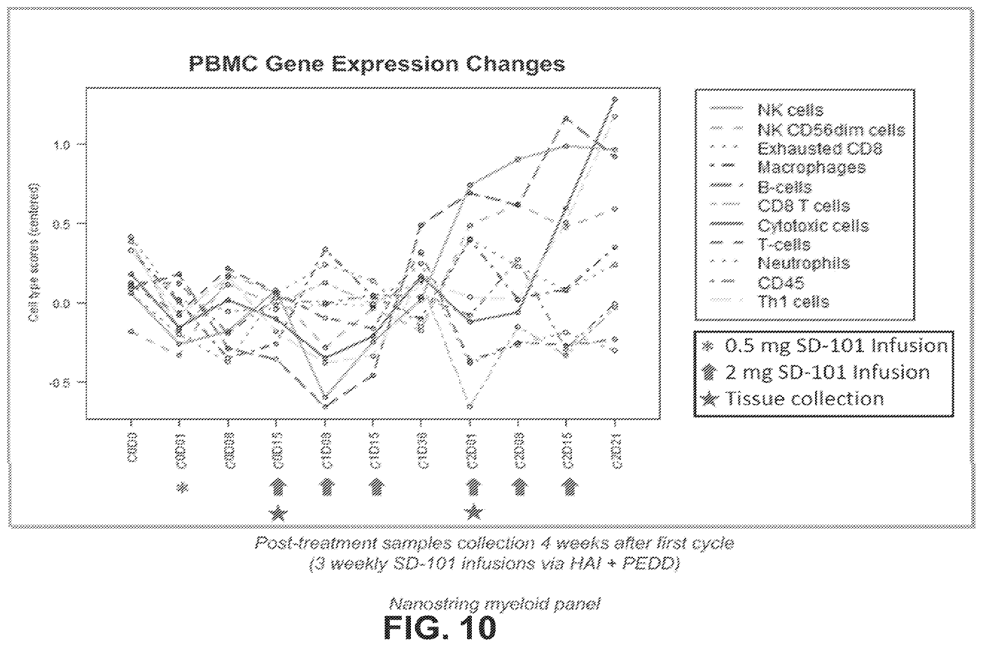

Post-treatment normal liver tissue samples (collected four weeks after the first cycle of three weekly SD-101 infusions via HAI and PEDD) from these patients were tested in a NanoString® myeloid cell panel to profile the expression of gene signatures for myeloid cells, including cytotoxic cells, DC, CD45, macrophages, T-cells, mast cells, neutrophils, NK cells, exhausted CD8, Th1 cells, and CD8 T cells. Data from the NanoString® myeloid cell panel is shown in FIG. 9 where BL-T refers to baseline time and D57-T refers to a time 4 weeks after the first cycle of 3 weekly infusions. As illustrated in FIG. 9, a NanoString® myeloid panel of post-treatment normal liver tissue samples (collected four weeks after the first cycle of three weekly SD-101 infusions via HAI and PEDD) showed SD-101 infusion was associated with decreases in Th1 cells, CD8 T cells, and cytotoxic cells in normal liver parenchyma. The opposite changes were seen in tumor cells. A lack of immune cell expansion/activation supports safety with respect to the effect of SD-101 on normal liver tissues. It's hypothesized that SD-101 treats immune dysfunction in the liver and tumor microenvironment, promoting recruitment of activated T cells from the normal liver into “cold” tumors. In other words, the normal liver tissue NanoString® data of FIG. 9 suggests movement of intrahepatic immune cells.

Post-treatment peripheral blood mononuclear cell (PBMC) samples from these patients were tested in a NanoString® myeloid cell panel to profile the expression of gene signatures for myeloid cells, including NK cells, NK CD56dim cells, exhausted CD8, macrophages, B-cells, CD8 T cells, cytotoxic cells, T-cells, neutrophils, CD45, and Th1 cells. Data from the NanoString® myeloid cell panel is shown in FIG. 10. FIG. 10 illustrates a NanoString® myeloid panel of post-treatment peripheral blood mononuclear cell (PBMC) samples. In this regard, there was a single 0.5 mg infusion of SD-101 followed by two cycles of three weekly 2 mg SD-101 infusions, with tissue collections occurring at the first weeks of each of the two cycles. As depicted in the figure, SD-101 hepatic artery infusions with PEDD were associated with peripheral blood increase in NK cells, Th1 cells, B cells, CD8 T cells, and cytotoxic cells. These increases were noted after the favorable immune changes within liver metastases, which included delivery of doses four through six of the second cycle. It's hypothesized that SD-101 treats immune dysfunction in the liver and tumor microenvironment, promoting emigration of activated dendritic cells and other immune cells from the reprogrammed liver. These PBMC gene expression changes suggest systemic activity following liver directed SD-101 infusions.

In summary, SD-101 and PEDD offers a tumor microenvironment modulation approach addressing an important immune-suppressive target (MDSC) with additional broad CPI enabling effects, while treating immune dysfunction in the surrounding non-tumor liver.

### Example 4

In Example 4, the impact of delivery of various TLR agonists (TLR4, TLR7, class B TLR9 (TLR9B) agonist, and class C TLR9 (TLR9C) agonist) on myeloid cells was evaluated, in particular their differential effects on MDSC and potential to immunomodulate liver tumor microenvironments (TME) was investigated.

There are three classes of TLR9 agonists, with differential effects on plasmacytoid DC (pDC) IFNα production, B and NK cell activation, and other immune cell populations. TLR9A agonists stimulate plasmacytoid DCs (pDCs) to produce IFNα. TLR9B agonists activate B and NK cells. TLR9C have a broader immunologic effects. Specific delivery challenges and immunosuppressive pathways, including unique programming of myeloid derived suppressor cells (MDSC), may limit immunotherapy success in the liver.

The effect of the various TLR agonists were evaluated in vitro by flow cytometry using different types of cells obtained from healthy human donors and also using murine bone marrow (BM) MDSCs.

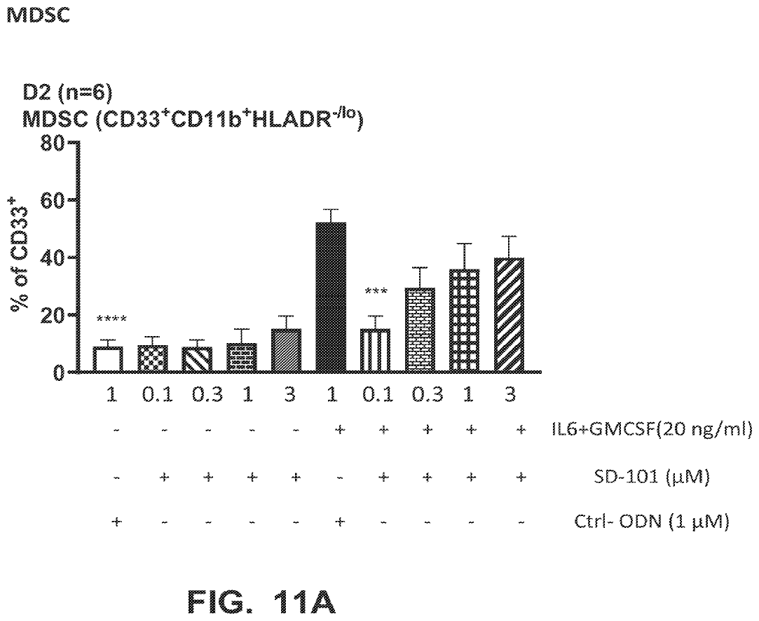

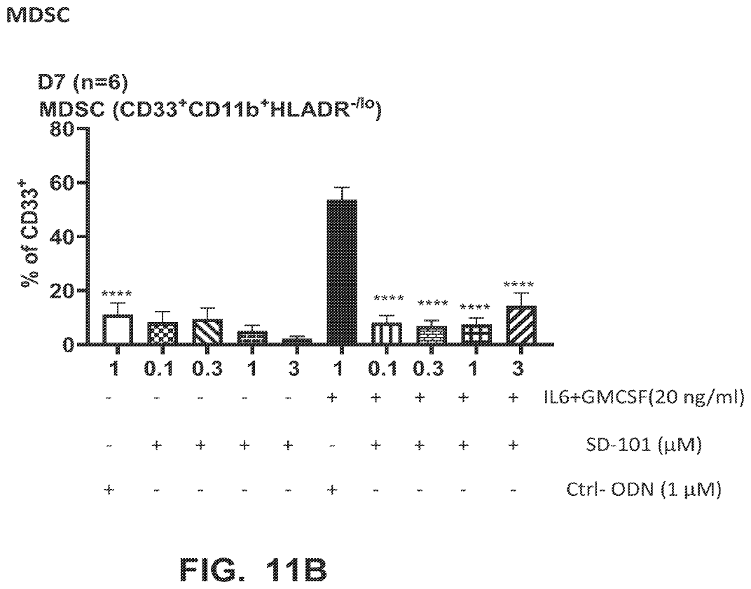

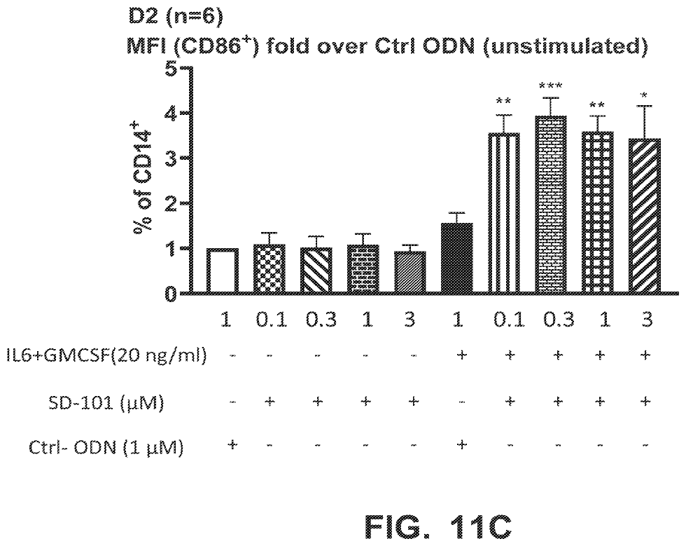

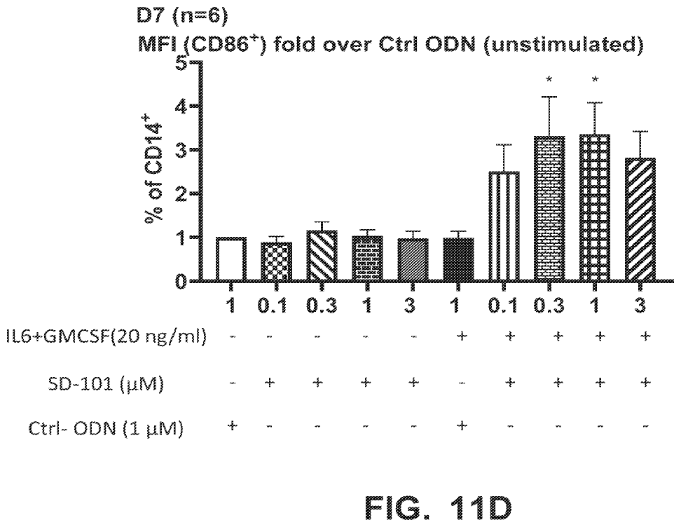

Peripheral Blood Mononuclear Cells (PBMCs) obtained from healthy human donors were cultured in vitro in the presence of or in the absence of IL6+GMCSF (20 ng/ml). The presence of IL6+GMCSF is used to induce MDSCs. The treated cells were then evaluated using flow cytometry to determine the effect of SD-101 on MDSCs (e.g., CD33⁺CD11b⁺HLADR^{-/lo}) and M1-Macrophage (e.g., CD86⁺). The data generated by flow cytometry with these cells are provide in FIGS. 11A to 11D.

FIGS. 11A to 11D show that TLR9 stimulation with SD-101 inhibits MDSC generation and enhance M1 macrophages production. The data of FIGS. 11A to 11D were generated with human PBMCs treated with increasing concentration (0.1-3 μM) of SD-101 and Ctrl ODN5328 (1 μM) for 2 days (FIGS. 11A and 11C) and 7 days (FIGS. 11B and 11D) in the presence/absence of IL6+GMCSF (20 ng/ml each). In FIGS. 11A and 11B, the effect of treatment with SD-101 or Ctrl ODN5328 on the MDSC population (CD33⁺CD11b⁺HLADR^{-/lo}) was evaluated. In FIGS. 11C and 11D, the effect of treatment with SD-101 or Ctrl ODN5328 on M1 macrophage (CD14⁺CD86⁺) was evaluated. As shown in FIGS. 11A-11D, a 0.3 μM SD-101 dose provided inhibition of MDSC generation and increase in M1 macrophages production after 7 days of treatment. 1-Way ANOVA followed by Tukey's post-hoc test was performed on the data show in FIGS. 11A to 11D where * indicates p<0.05, ** indicates p<0.01, *** indicates p<0.001, and **** indicates p<0.0001 vs. IL6+GMCSF stimulated 1 μM Ctrl ODN treated cells, n=number of healthy donors.

### Example 5

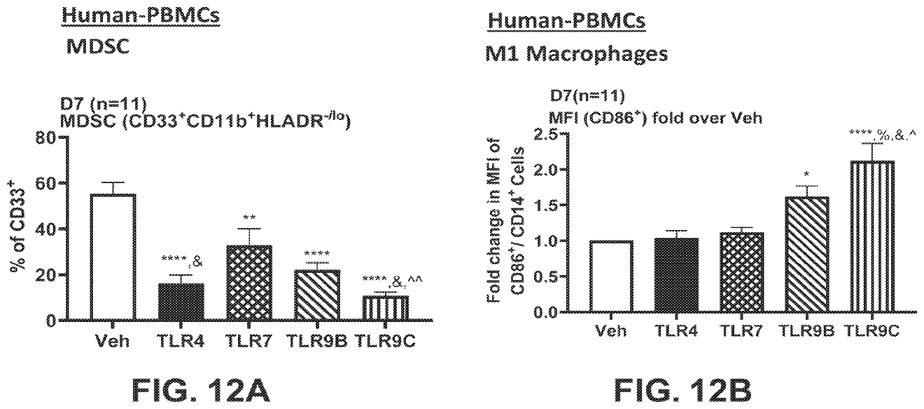

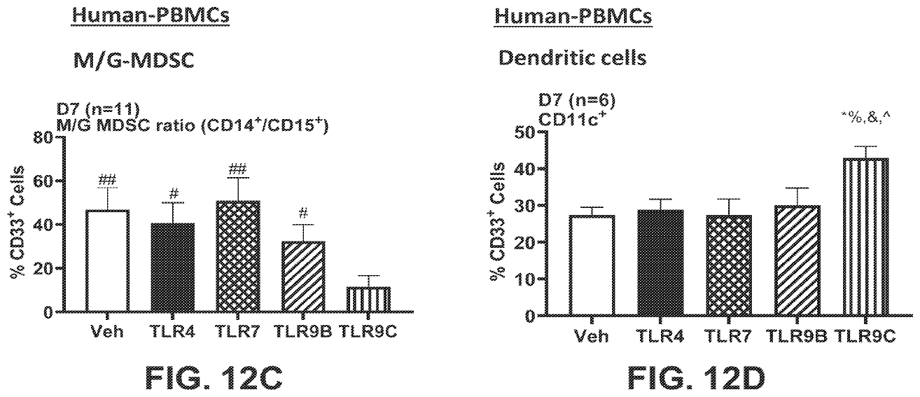

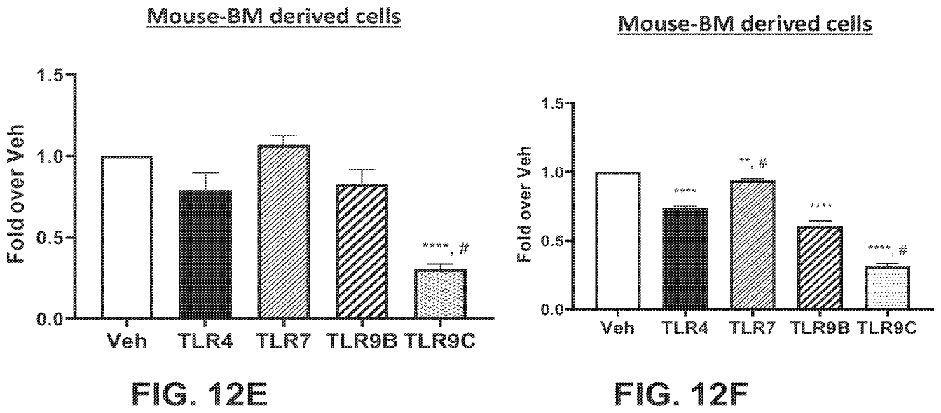

In Example 5, PBMCs obtained from healthy human donors were cultured in vitro in IL6+GMCSF (20 ng/ml) to induce MDSCs, and subsequently treated with a TLR agonist. Flow cytometry was performed to evaluate the effects of different TLR agonists on MDSCs (CD33⁺CD11b⁺HLADR⁻), subtypes of MDSCs (monocytic/granulocytic CD14⁺/CD15⁺MDSCs), M1 macrophages (CD14⁺CD86⁺), and monocytic dendritic cells (CD14⁺CD11c⁺CD123). The TLR agonists evaluated include a TLR4 agonist, a TLR7 agonist, a class B TLR9 agonist, and SD-101, which is a class C TLR9 agonist. In addition, bone marrow (BM) murine MDSCs (CD11b⁺GR-1⁺) were treated for 72 h with GMCSF and effects of different TLR agonists on murine BM MDSCs were evaluated by flow cytometry. Data generated from flow cytometry of the different types of human and murine cells are provided in FIGS. 12A to 12F.

FIGS. 12A to 12F show the effects of different TLR agonists on human MDSCs and other immune cells (i.e., murine immune cells). The data of FIGS. 12A to 12D are generated using PBMCs obtained from healthy human donors that have been stimulated with IL6+GMCSF (20 ng/ml each) and treated with vehicle or 0.3 μM of a TLR4 agonist, a TLR7 agonist, a class B TLR9 agonist, or SD-101, which is a class C TLR9 agonist for 7 days. In FIG. 12A, the effects of treatment with different TLR agonists on MDSCs were evaluated. In FIG. 12B, the effects of treatment with different TLR agonists on M1 macrophages were evaluated. In FIG. 12C, the effects of treatment with different TLR agonists on M/G-MDSCs were evaluated. In FIG. 12D, the effects of treatment with different TLR agonists on dendritic cells (CD33⁺CD11c⁺) were evaluated.

Additionally, FIGS. 12E and 12F show the effects of different TLR agonists on murine BM MDSCs. The data of FIG. 12E are generated using cells obtained from mouse bone marrow (BM) treated in the absence of murine GMCSF. The data of FIG. 12F are generated using cells obtained from mouse bone marrow (BM) treated in the presence of murine GMCSF. Students' t test and 1-Way ANOVA followed by Tukey's post-hoc test was performed on the data shown in FIGS. 12A to 12F where * indicates p<0.05, ** indicates p<0.01, *** indicates p<0.001, and **** indicates p<0.0001 vs. Veh. In addition, % indicates p<0.01 vs. TLR4, & indicates p<0.01 vs. TLR7, \hat{ } indicates p<0.001 vs. TLR9B, # indicates p<0.05, and ## indicates p<0.01 vs SD-101.

As shown in the data of FIGS. 12A to 12F, SD-101, a TLR9C agonist, significantly inhibited MDSC expansion compared to vehicle (Veh), TLR9B, TLR4 and TLR7 agonists on day 2 (p<0.01; n=11). Similar results were obtained with murine BM derived MDSCs (p<0.001; n=5). SD-101 significantly reduced the human M/G-MDSC ratio as compared to Veh, TLR4, TLR7 and TLR9B agonists (p<0.05; n=11). In addition, SD-101 shifted dendritic cells towards a myeloid program compared to Veh, TLR4 and TLR7 agonists (p<0.05; n=6). In summary, SD-101 significantly inhibited MDSC expansion compared to vehicle (Veh), TLR9B, TLR4 and TLR7 agonists on day 2, and that SD-101 significantly reduced the human M/G-MDSC ratio as compared to Veh, TLR4, TLR7 and TLR9B agonists.

### Example 6

In Example 6, PBMCs obtained from healthy human donors (n=6) were cultured in vitro in IL6+GMCSF (20 ng/ml) to induce MDSCs, and subsequently treated with either a TLR9B agonist or SD-101, which is a TLR9C agonist, at a concentration of 0.3 μM for 2 days. Total RNA for each of half of the samples (n=3) were isolated and evaluated using NanoString® analysis. The samples for the NanoString® analysis were also analyzed by qRT-PCR for verification. The other half of the samples were evaluated using flow cytometry to determine the effects of the TLR9B agonist and SD-101 on MDSCs (CD33⁺CD11b⁺HLADR^{-/lo}) and M1 macrophages (CD14⁺CD86⁺).

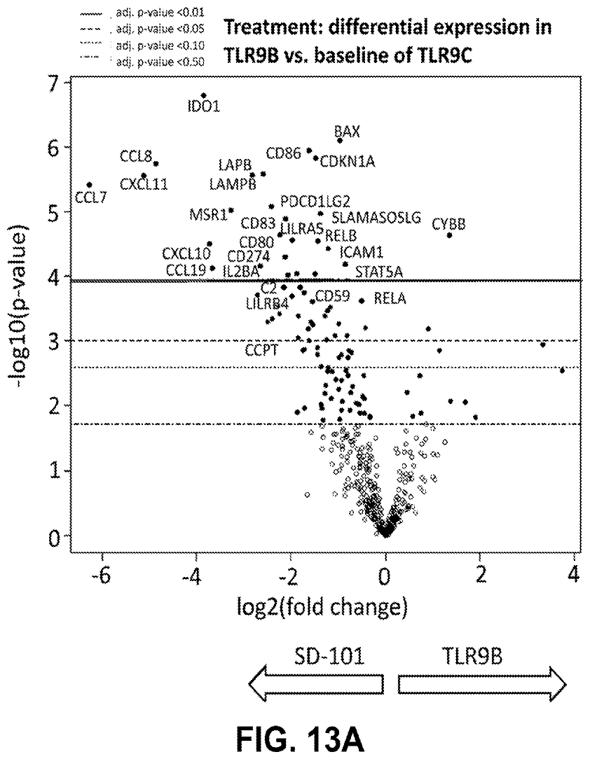

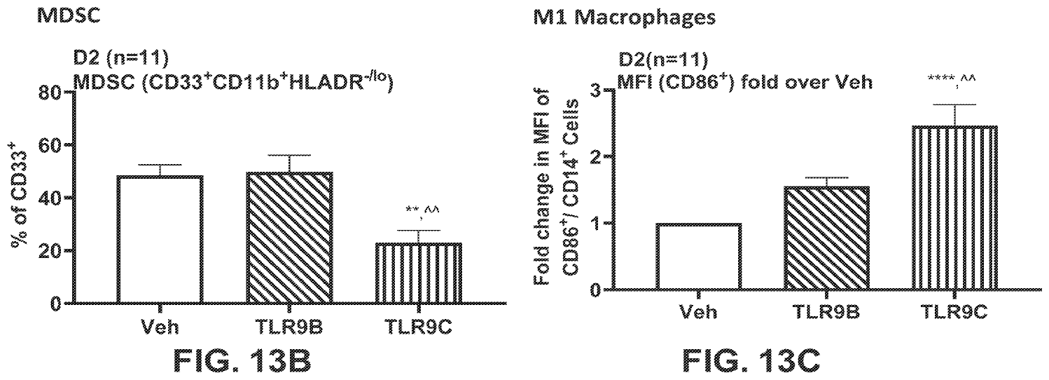

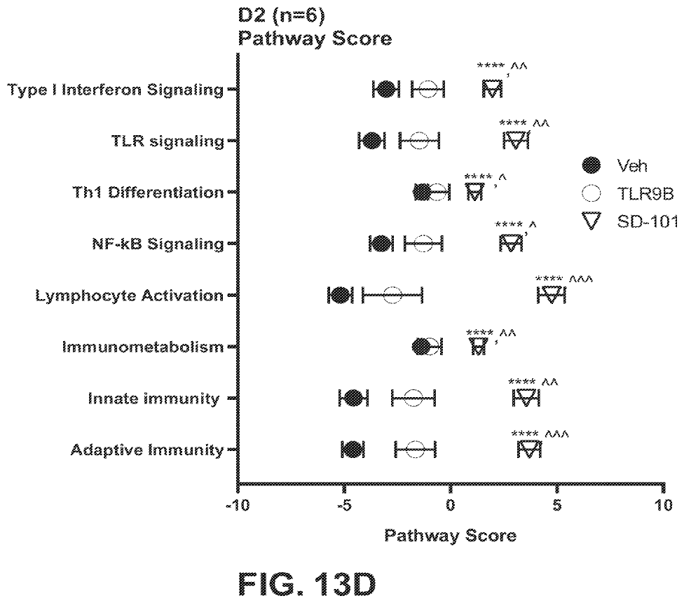

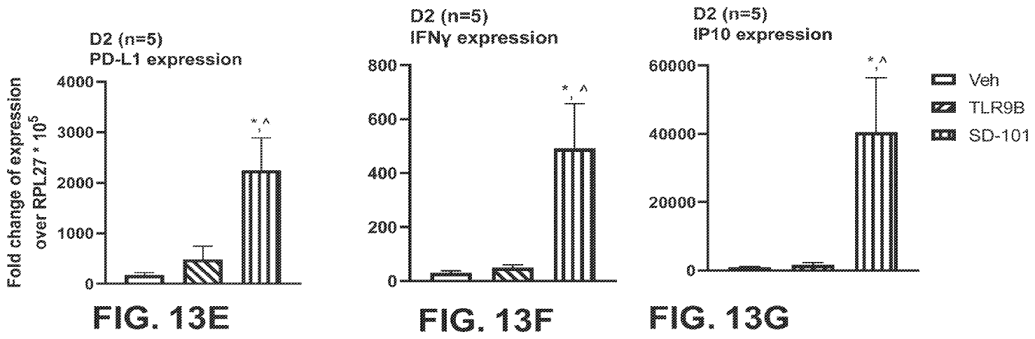

FIG. 13A to 13G demonstrate that TLR9 stimulation with TLR9C agonist SD-101 promotes enhanced pro-inflammatory and anti-tumorigenic signaling compared to TLR9B agonists. Specifically FIG. 13A shows a volcano plot illustrating gene expression of human PBMCs after treatment with TLR9B agonist vs. SD-101 (TLR9C agonist) for 2 days, as determined using NanoString® mRNA analysis for the samples prepared as described above.

Table 2 shows data generated using NanoString® analysis demonstrating that SD-101 up regulates numerous genes more than TLR9B agonist.

TABLE 2
SD-101 upregulates more than TLR9B agonist

| | Log₂ fold | |
|---|---|---|
| Genes | change | P-value |
| CCL7-mRNA | 6.26 | 3.75 × 10⁻⁶ |
| CXCL11-mRNA | 5.1 | 2.53 × 10⁻⁶ |
| CCL8-mRNA | 4.88 | 1.82 × 10⁻⁶ |
| IDO1-mRNA | 3.86 | 1.51 × 10⁻⁷ |
| CCL19-mRNA | 3.67 | 7.80 × 10⁻⁵ |
| CXCL 10-mRNA | 3.66 | 3.32 × 10⁻⁵ |
| MSR1-mRNA | 3.29 | 9.93 × 10⁻⁶ |
| LAMP3-mRNA | 2.76 | 3.13 × 10⁻⁶ |
| TNFRSF9-mRNA | 2.71 | 0.000197 |
| TNFRSF11A-mRNA | 2.61 | 2.95 × 10⁻⁶ |
| IL2RA-mRNA | 2.61 | 7.58 × 10⁻⁵ |
| FAS-mRNA | 2.52 | 8.01 × 10⁻⁵ |
| LAG3-mRNA | 2.49 | 0.000501 |
| PD-L2 mRNA | 2.41 | 8.04 × 10⁻⁶ |
| CCL2-mRNA | 2.41 | 0.00046 |
| IRF4-mRNA | 2.29 | 0.000273 |
| CD80-mRNA | 2.25 | 2.37 × 10⁻⁵ |
| IFIT2-mRNA | 2.25 | 0.000386 |
| PD-L1-mRNA | 2.16 | 5.02 × 10⁻⁵ |
| CD83-mRNA | 2.12 | 1.28 × 10⁻⁵ |

Table 3 shows data generated using NanoString® analysis demonstrating that SD-101 down regulates other genes more than TLR9B agonist.

TABLE 3
SD-101 down regulates more than TLR9B agonist

| Genes | Log2 fold change | P-value |
|---|---|---|
| MRC1-mRNA | −1.14 | 0.00142 |
| CCR2-mRNA | −1.26 | 0.0364 |
| CYBB-mRNA | −1.35 | 2.37 × 10⁻⁵ |
| CCL24-mRNA | −1.38 | 0.00869 |
| CR1-mRNA | −1.68 | 0.00875 |
| IRAK3-mRNA | −1.89 | 0.0149 |
| MARCO-mRNA | −3.33 | 0.00114 |
| FN1-mRNA | −3.73 | 0.00283 |

In addition, FIGS. 13B and 13C show the effects of TLR9B and TLR9C agonists on MDSCs and M1 macrophages, respectively, as determined by flow cytometry for the samples prepared as described above. FIG. 13D shows the pathway scores for immunoregulatory pathways affected by TLR9B vs. SD-101 as determined by nSolver.4 software using data generated from NanoString® analysis. FIGS. 13E, 13F and 13G shows qRT-PCR data of the same samples analyzed in FIG. 13A. The data of FIGS. 13E, 13F and 13G show superior effects of SD-101 in enhancing the PD-L1, IFNγ and IP-10 expressions.

Multiple t-test was performed for pathway score analysis shown in FIG. 13D. 1-Way ANOVA followed by Tukey's post-hoc test was performed for qRT-PCR analysis shown in FIGS. 13E-13G. For both FIGS. 13B to 13E-13G * indicates p<0.05, and ***** indicates p<0.0001 vs. Veh. Additionally, \hat{ } indicates p<0.05, \hat{ }\hat{ } indicates p<0.01, and \hat{ }\hat{ }\hat{ } indicates p<0.001 vs. TLR9B.

As shown above, NanoString® gene expression analysis revealed that SD-101 induced higher adaptive immunity, innate immunity, immunometabolism scores compared with Veh and TLR9B agonist (p<0.05; n=3). There was greater induction of TLR, Th1, NFκB and lymphocyte activation signatures in SD-101 as compared to Veh and TLR9B agonist (p<0.05; n=3). SD-101 induced more PD-L1, IFNγ and IP10 gene expression compared to Veh and TLR9B (p<0.05; n=6), in addition to B and NK cell expansion compared to Veh (p<0.05; n=6). In summary, there was greater induction of innate, adaptive immune signaling pathways, TLR, Th1, NFκB and lymphocyte activation signatures in SD-101 as compared to Veh and TLR9B agonist, and SD-101 induced more PD-L1, IFNγ and IP10 gene expression as compared to Veh and TLR9B agonist.

Overall, SD-101 inhibited MDSC expansion and enhanced polarization towards M1 macrophages. The favorable impact on MDSC, in addition to broad immune activating effects, suggests that TLR9C agonists, if delivered effectively, have the potential to enable better performance of other immunotherapy agents within the hostile liver tumor microenvironments (TMEs).

### Example 7

In Example 7, infusions of a class C TLR9 agonist, SD-101, were administered to a human patient via pressure-enabled hepatic artery infusion (PEDD/HAI) using the TriNav® infusion system, with the aim of enhancing response rates to CPI therapy in human patients with uveal melanoma liver metastases, advanced hepatocellular carcinoma, or advanced intrahepatic cholangiocarcinoma. The study included Cohorts A, B, and C. Cohort A is administered SD-101 as a single agent (where n=9), Cohort B is administered SD-101 via TriNav+systemic administration of anti-PD-1 (i.e., nivolumab or pembrolizumab) (where n=6-12), and Cohort C is administered SD-101 via TriNav+systemic administration of anti-PD-1 (i.e., nivolumab)+systemic administration of anti-CTLA-4 (i.e., ipilimumab) (where n=6-12). The amount of SD-101 administered to human patients in Cohorts A, B and C may be across a dose escalation range from about 0.5 mg to about 8 mg per dose (i.e., 0.5 mg, 2 mg, 4 mg and 8 mg). The dosing regimen includes administering SD-101 via PEDD over two cycles, with each cycle comprising three weekly doses of SD-101 (i.e. administration of SD-101 once per week over three consecutive weeks) and a rest period of five weeks following the weekly administration of SD-101.

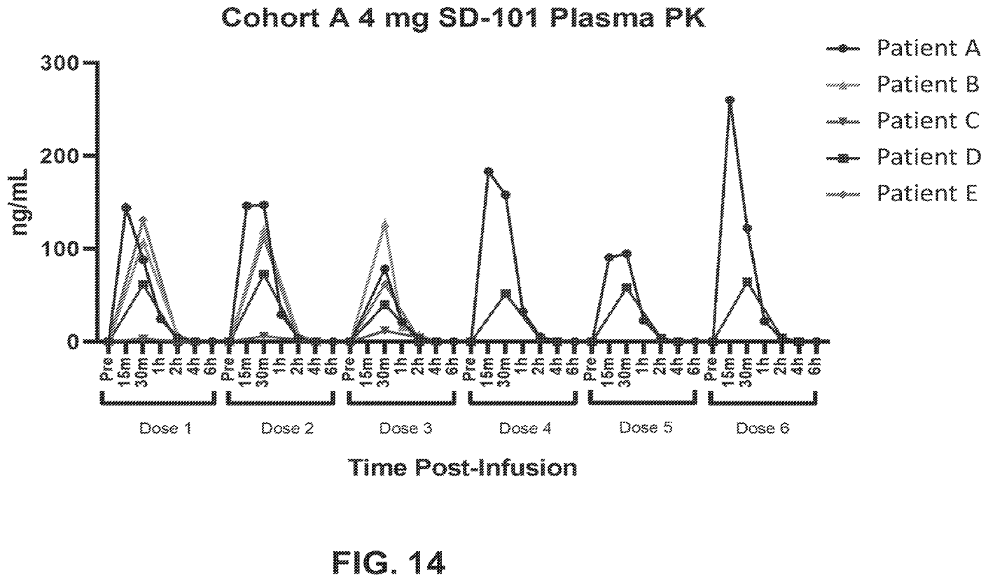

FIG. 14 illustrates the pharmacokinetic profile of SD-101 levels in plasma of five different patients in Cohort A, following 4 mg dosing of single agent SD-101 for six hours post-infusion for each of six doses administered over two cycles as discussed above. Specifically, as depicted in the figure, following infusion with TriNav, there were transient levels (<2 hrs) of SD-101 detected in plasma following infusion with TriNav. At all doses (not shown), the levels of systemic exposure detected were transient (<4 hours).

High SD-101 levels were observed in liver of patients following infusion of SD-101 with TriNav. In patients who were administered SD-101 at 8 mg per dose, SD-101 was detected in liver tissue up to 2340 ng/ml. This human clinical data demonstrate that TriNav can be used to achieve higher liver SD-101 levels with limited systemic exposure.

Additionally, following SD-101 treatment, PMBC cells from patients administered SD-101 in the manner describe above were analyzed by NanoString® for gene expression levels at multiple time points. Specifically, NanoString® analysis of gene expression levels in liver metastases from three patients in the 2 mg SD-101 single agent cohort revealed increases in ISG15, IL-9, IFNα, and IL-2 transcripts and decreases in ARG1 and IDO transcripts, with increased scores for macrophages, exhausted CD8 T cells, Th1 cells, and Th1 activation. The NanoString® gene expression data showed that SD-101 delivered via TriNav was associated with evidence of expansion of anti-tumor natural killer cells in the blood, which is consistent with systemic immune activation.

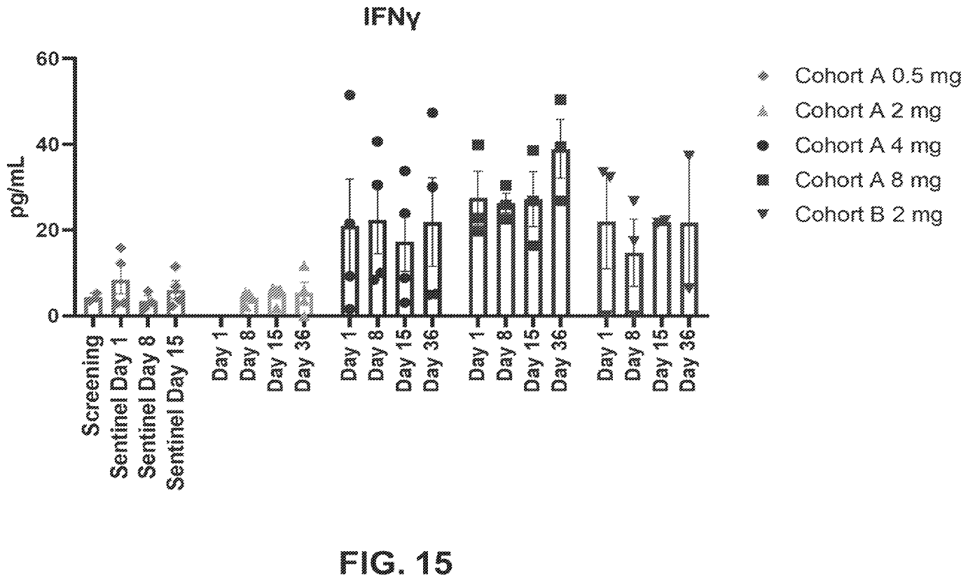

A dose response trend was noted in serum cytokines with increase across the 2 mg, 4 mg, and 8 mg single-agent dose levels for IL-18, IFNγ, IP-10, and soluble CD25. In particular, FIG. 15 illustrates changes in serum IFNγ following increasing dosing levels of single agent SD-101 (Cohort A), and SD-101 in combination with a CPI (i.e., nivolumab or pembrolizumab) (Cohort B) after each of three doses administered over a first cycle. Specifically, the figure depicts a trend of increasing serum IFNγ levels following infusion of SD-101 via TriNav for Cohort A (at doses of 0.5 mg, 2 mg, 4 mg, and 8 mg of SD-101) and Cohort B (at 2 mg dose of SD-101, in combination with pembrolizumab or nivolumab). The serum IFNγ were determined by Luminex assays. The data of FIG. 15 demonstrates SD-101 infusion with TriNav results in IFNγ cytokine induction.

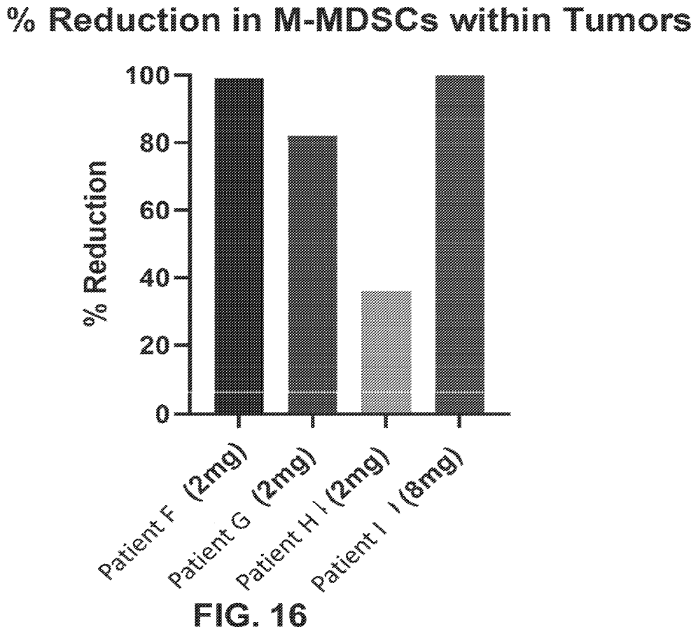

FIG. 16 illustrates changes in monocytic MDSC concentrations within tumors following varying dosing levels of single agent SD-101 (Cohort A) in the manner described above. Specifically, the figure depicts a reduction in monocytic MDSC concentrations within the tumors for (i) three patients who were administered 2 mg doses of SD-101 and (ii) one patient who was administered 8 mg doses of SD-101. The monocytic MDSC concentration were determined by multiplex immunofluorescence microscopy. The % reduction in monocytic MDSC concentrations within tumors were calculated from earliest available time point (Day 1 or Day 57) to latest available time point (Day 57 or Day 100). The data of FIG. 16 demonstrates SD-101 infusion with TriNav results in MSDC elimination as shown in decreases in liver tumor monocytic MDSC levels.

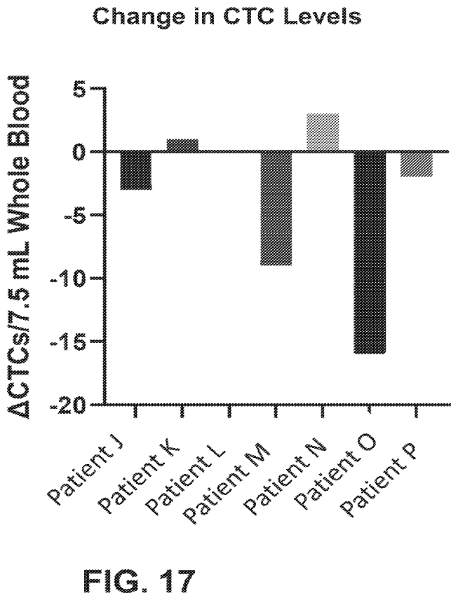

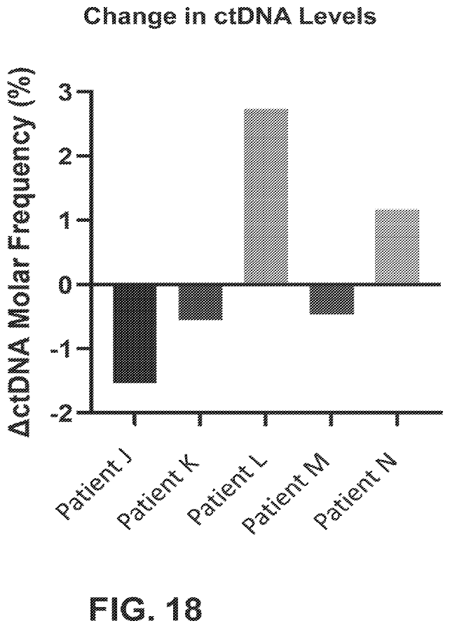

For patients who received 2 mg SD-101⁺CPI with available liquid biopsy data, 4 of 7 demonstrated decreases in circulating tumor cells after 1 cycle of treatment, with 3 of 5 showing circulating tumor DNA (ctDNA) decreases after the first cycle, as shown below in FIGS. 17 and 18.

FIG. 17 illustrates changes in circulating tumor cells following 2 mg dosing of SD-101 with CPI after the first cycle of treatment. Specifically, the figure depicts that four out of seven patients had circulating tumor cell levels decrease following 2 mg dosing of single agent SD-101, thereby resulting in a decrease of the tumor burden.

FIG. 18 illustrates changes in circulating tumor DNA (ctDNA) following 2 mg dosing of single agent SD-101 after the first cycle of treatment. Specifically, the figure depicts that three out of five patients had circulating tumor DNA (ctDNA) levels decrease following 2 mg dosing of single agent SD-101, thereby resulting in a decrease of the tumor burden. The data provided in FIG. 18 are for the same first 5 patients as FIG. 17.

### Example 8

In Example 8, the TLR9C agonist, SD-101 delivered via PEDD in combination with anti-PD-1 antibody delivered SQ or IP significantly reduced LM in mice. Further analysis of tumor infiltrating lymphocytes (CD45+ cells) isolated from the tumor-bearing livers revealed that anti-PD-1 in combination with SD-101 significantly reduced liver MDSCs. The percentage of B cells; CD3+ T cells and the ratio of M1/M2 macrophages increased significantly as compared to Veh. There were no significant differences between SQ and IP CPI delivery in controlling tumor progression or modulation of the tumor microenvironment. This data demonstrates that TLR9 stimulation via PEDD in combination with anti-PD-1, irrespective of the route of delivery, provided equal control of LM. Also, PEDD of a class C TLR9 agonist has the potential to prime the TME to reduce immunosuppression in LM which may improve the anti-tumor efficacy of anti-PD-1 irrespective of the route of administration. In some examples, the checkpoint inhibitors are administered systemically or subcutaneously before the administration of the TLRC agonist.

In this example, SD-101 was delivered in a murine model of PEDD in combination with an anti-PD-1 antibody administered either intraperitoneally (IP) or subcutaneously (SQ) to evaluate whether the route of CPI administration impacts the ability of intrahepatic TLR9 stimulation to control liver metastasis. To develop liver metastasis, C57/BL6 mice were challenged with MC38-Luc tumor cells via the intra-splenic route followed by splenectomy. After a week, mice were treated with 10 μg SD-101 via PEDD and twice weekly anti-PD-1 antibody delivered either IP or SQ. Tumor burden was monitored by IVIS and on D10 liver was harvested to isolate CD45⁺ cells. Myeloid-derived suppressive cells (MDSCs) blunt the activity of immunotherapy through the promotion of an immunosuppressive tumor microenvironment (TME) in the setting of liver metastases. Flow cytometry analysis was performed to quantify MDSCs (CD11b⁺Gr1⁺), B cells (B220), T (CD3⁺) cells, M1(F4/80⁺CD38⁺Egr2⁻), and M2 (F4/80⁺CD38⁻ Egr2⁺) macrophages in the TME.

SD-101 delivered via PEDD in combination with anti-PD-1 antibody delivered either via SQ or IP significantly (p<0.0001) reduced liver metastasis progression (fold over DO) compared to control (Veh:87.46 vs. SD-101: 13.90 vs. SQ: 0.002 vs. IP: 0.04). Flow cytometry analysis of CD45⁺ cells isolated from the tumor-bearing livers revealed that CPI in combination with SD-101 significantly reduced liver MDSCs (Veh: 37.57% vs. SQ: 7.18% vs. IP: 10.18%; p<0.05). The percentage of B cells (Veh: 8.32% vs. SQ: 18.09% vs. IP: 15.65%: p<0.05); T cells (Veh: 7.14% vs. SD-101: 15.18% vs. SQ: 17.81% vs. IP: 19.13%: p<0.05) and the ratio of M1/M2 macrophages (Veh: 2.25 vs. SD-101: 12.8 vs. SQ: 12.99 vs. IP: 12.90: p<0.05) increased significantly as compared to Vehicle (Veh). There were no significant differences between SQ and IP CPI delivery in controlling tumor progression or modulation of the TME.

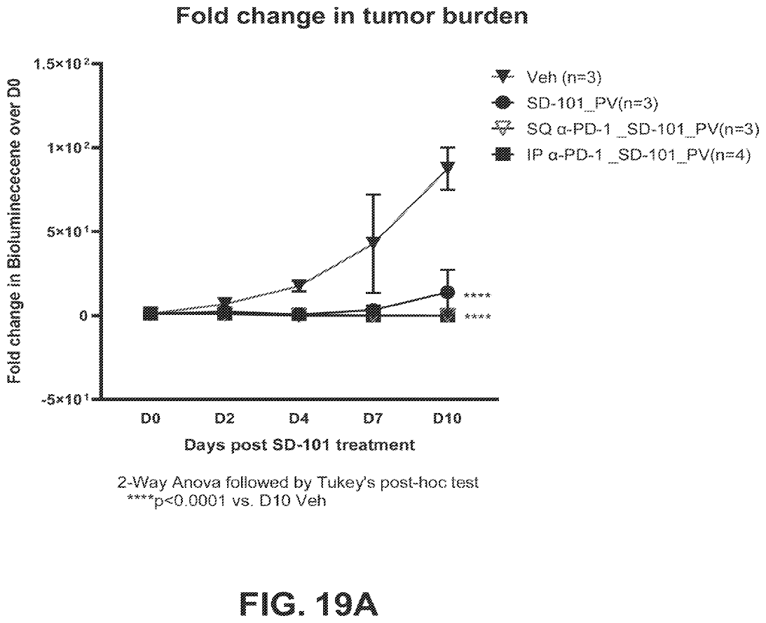

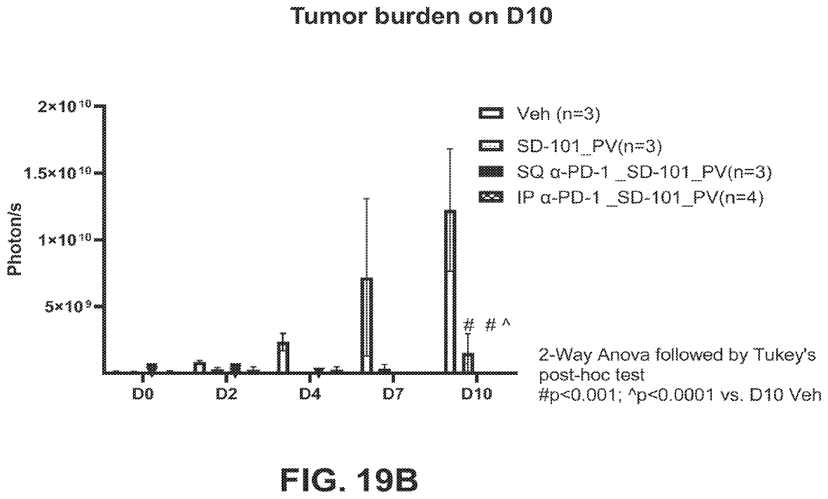

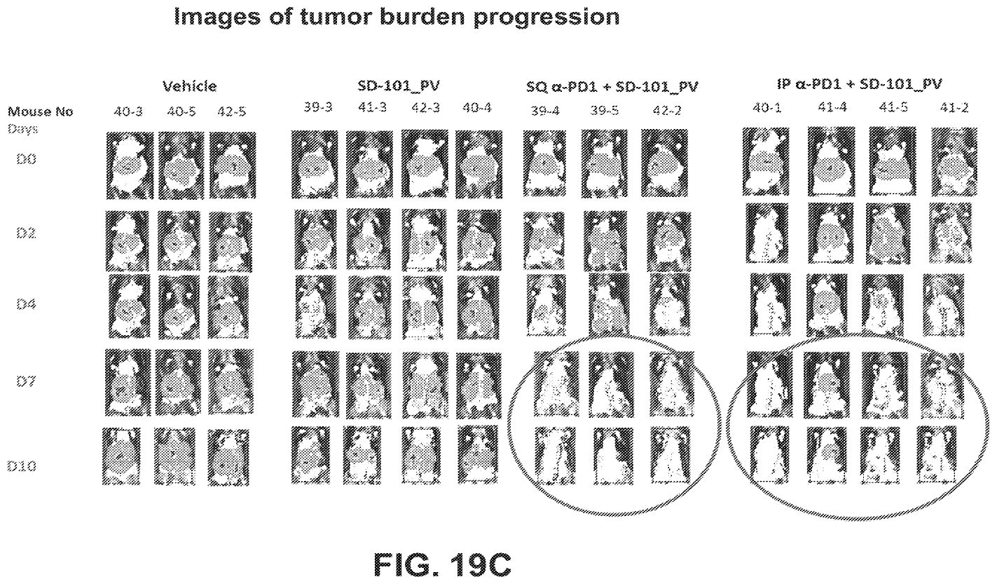

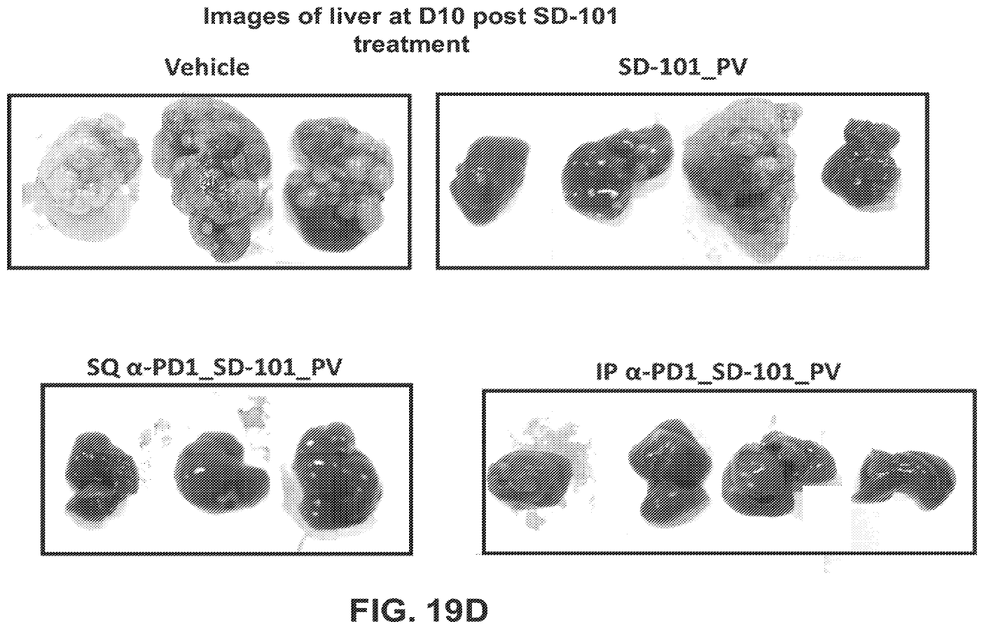

FIG. 19A illustrates a change in the tumor progression of the mouse liver metastasis in response to PEDD administration of SD-101 in combination with anti-PD-1 antibody delivered either IP or SQ as described in this Example. C57/BL6 mice were challenged with MC38-Luc tumor cells via the intra-splenic route followed by splenectomy to develop LM. After a week, mice were treated with 10 g SD-101 via PEDD and with anti-PD-1 antibody on DO, D2, D4 and D7 delivered either IP or SQ. Tumor burden was monitored by in vivo imaging system (IVIS) and fold changes over DO tumor burden has been reported. 2-Way ANOVA followed by Tukey's post-hoc test was performed on the data show in FIG. 19A where **** indicates p<0.001 vs. D10 Veh.

FIG. 19B illustrates changes in the tumor burden of the mouse liver metastasis in response to PEDD administration of SD-101 in combination with anti-PD-1 antibody delivered either IP or SQ as compared to Vehicle at 10 days after treatment. IVIS value has been reported as photon/s. 2-Way ANOVA followed by Tukey's post-hoc test was performed on the data show in FIG. 19B where # indicates p<0.001 vs. D10 Veh and \hat{ } indicates p<0.0001 vs. D10 Veh.

FIG. 19C provides images captured by the IVIS over different time points after initiating treatment by PEDD administration of SD-101 in combination with anti-PD-1 antibody delivered either IP or SQ as compared to Vehicle. FIG. 19D provides images captured of harvested liver at after 10 days of treatment with PEDD administration of SD-101 in combination with anti-PD-1 antibody delivered either IP or SQ as compared to Vehicle, according to Example 8.

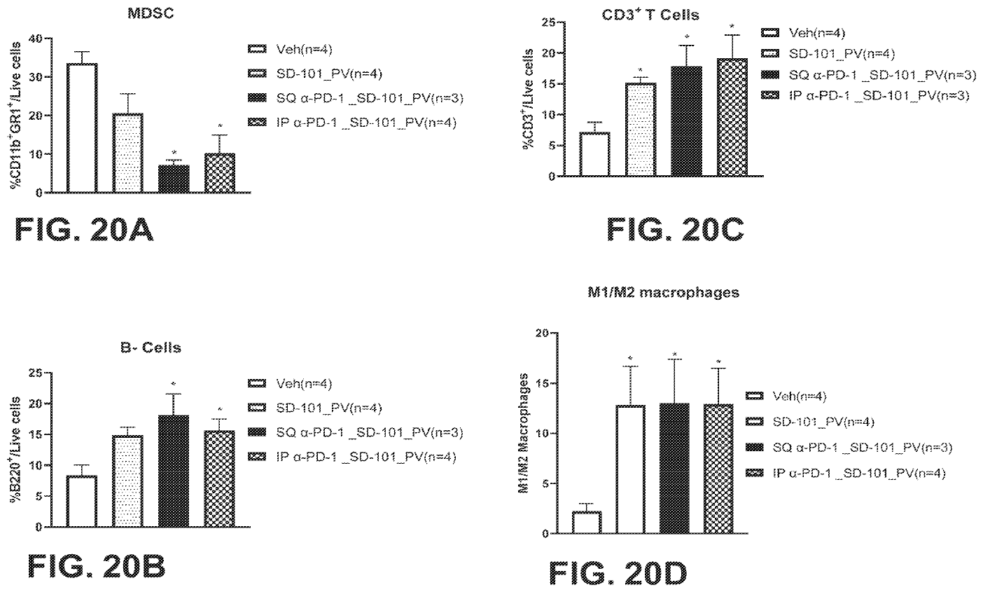

FIG. 20A illustrates changes in MDSCs present in the LM in response to PEDD administration of SD-101 in combination with anti-PD-1 antibody delivered either IP or SQ as compared to Vehicle at 10 days after treatment, as described in this Example. On Day 10 (D10), liver was harvested to isolate CD45⁺ cells. In particular, Liver was harvested on D10, following collagenase digestion NPC was isolated; and CD45⁺ cells were isolated using magnetic bead. Flow cytometry (FC) was performed using antibodies against: CD11b; GR1, NK1.1, B220, PD-L1. The FC analysis was performed to quantify MDSCs (CD11b⁺Gr1⁺).

FIG. 20B illustrates changes in the B (B220⁺) cells of the isolated CD45⁺ cells from liver metastasis in response to PEDD administration of SD-101 in combination with anti-PD-1 antibody delivered either IP or SQ as compared to Vehicle at 10 days after treatment. FIG. 20C illustrates changes in the T (CD3⁺) cells of the isolated CD45⁺ cells from liver metastasis in response to PEDD administration of SD-101 in combination with anti-PD-1 antibody delivered either IP or SQ as compared to Vehicle at 10 days after treatment. FIG. 20D illustrates changes in the M1 and M2 macrophages of the isolated CD45⁺ cells from liver metastasis. M1(F4/80⁺CD38⁻Egr2⁻), and M2 (F4/80-CD38⁻Egr2⁺) macrophages in the tumor microenvironment, as quantified by FC, in response to PEDD administration of SD-101 in combination with anti-PD-1 antibody delivered either IP or SQ as compared to Vehicle at 10 days after treatment. In view of the data of Example 8 described above, SD-101 administered via PEDD in combination with CPI that was delivered IP or SQ provided control of liver metastasis, with intrahepatic TLR9 stimulation enabling CPI via either route equally. It is believed that PEDD of a class C TLR9 agonist may prime the TME to reduce immunosuppression in liver metastasis which may improve the anti-tumor efficacy of CPIs irrespective of the route of administration.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. A method for treating liver immune dysfunction comprising administering to a subject in need thereof a therapeutically effective amount of a toll-like receptor 9 agonist having the structure: 5′-TCG AAC GTT CGA ACG TTC GAA CGT TCG AAT-3′ (SEQ ID NO: 1), wherein the liver immune dysfunction is induced by at least one tumor resulting from metastasis in the liver or at least one primary liver cancer.

2. The method of claim 1, wherein the TLR9 agonist is administered through a device by hepatic arterial infusion (HAI).

3. The method of claim 1, wherein the TLR9 agonist is administered through a device by portal vein infusion (PVI).

4. The method of claim 1, wherein the therapeutically effective amount of the TLR9 agonist administered is in the range of about 0.01-20 mg.

5. The method of claim 4, wherein the therapeutically effective amount of the TLR9 agonist administered is selected from the group consisting of 2 mg, 4 mg, or 8 mg.

6. The method of any one of claims 1-5, wherein the TLR9 agonist may be administered through a catheter device.

7. The method of claim 6, wherein the catheter device comprises a one-way valve that responds dynamically to local pressure and/or flow changes.

8. The method of claim 6, wherein the TLR9 agonist is administered through the catheter device via pressure-enabled drug delivery.

9. The method of claim 6, wherein the TLR9 agonist is administered for a period of time of about 10-200 minutes.

10. The method of claim 9, wherein the TLR9 agonist is administered for a period of time of about 10-60 minutes.

11. The method of claim 10, wherein the TLR9 agonist is administered for a period of time of about 25 minutes.

12. The method of any one of claims 1-5, wherein the administration of the TLR9 agonist results in changes in gene expression within the metastasis in the liver.

13. The method of claim 12, wherein the changes in the gene expression include activation of the immune cells in normal liver tissue and migration of the activated immune cells into the at least one tumor.

14. The method of claim 12, wherein the changes in the gene expression include at least one of: increased TLR signaling; increased leukocytes; increased exhausted CD8 T cells; induction of Th1 programming; reduction of Th2 programming; increased T cell receptor and T cell co-stimulatory signaling; increased IL9, IL15, CCL7; B cell activation; induction of mast cells; induction of NK cells; induction of IFNγ; increased interferon signaling; increased chemokine signaling; increased IL6 in the liver without an increase in the blood; decreased M2 macrophages; increased M1 macrophages; decreased in MDSC; decreased angiogenesis and VEGF; and decreased fatty acid oxidation.

15. The method of any one of claims 1-5, wherein the TLR9 agonist is administered in combination with one or more checkpoint inhibitors.

16. The method of claim 15, wherein the checkpoint inhibitors are administered systemically, either concurrently, before, or after the administration of the TLR9 agonist.

17. The method of claim 16, wherein the one or more checkpoint inhibitors include at least one of nivolumab, pembrolizumab, and ipilimumab.

18. The method of claim 16, wherein the administration of the TLR9 agonist comprises a dosing regimen comprising cycles, in which one or more of the cycles comprise the administration of the TLR9 agonist via a catheter device by hepatic arterial infusion followed by the systemic administration of the one or more checkpoint inhibitors.

19. The method of claim 16, wherein the administration of the TLR9 agonist in combination with the one or more checkpoint inhibitors results in a decrease of one of circulating tumor cell levels and circulating tumor DNA levels.

20. The method of claim 15, wherein the one or more checkpoint inhibitors is administered intraperitoneally or subcutaneously.
